(19) United States
(12) Patent Application Publication
(10) Pub. No.: US 2010/0129361 A1
(43) Pub. Date: May 27, 2010

(54) IMMUNOSUPPRESSION WITH ANTIBODY AGAINST ITM2A

(75) Inventors: I-Cheng Ho, Newton, MA (US); Peter Kim, Los Angeles, CA (US); Sung-Yun Pai, West Roxbury, MA (US)

Correspondence Address: DAVID S. RESNICK, NIXON PEABODY LLP, 100 SUMMER STREET, BOSTON, MA, 02110-2131, US

(73) Assignee: THE BRIGHAM AND WOMEN'S HOSPITAL, Boston, MA (US)

(21) Appl. No.: 12/598,567

(22) PCT Filed: May 1, 2008
(86) PCT No.: PCT/US08/62146

§ 371 Date: Nov. 2, 2009

Related U.S. Application Data
(60) Provisional application No. 60/927,107, filed on May 1, 2007.

(51) Int. Cl. A61K 39/395; A61P 37/06

(52) U.S. Cl. 424/133.1; 424/173.1

(57) ABSTRACT

The invention relates to methods of T cell immune system modulation and the treatment of immune system related diseases and disorders. In particularly, embodiments of the invention provides immunotherapeutics in the form of antibodies, bioengmeered antibodies and recombinant proteins for the treatment of autoimmune diseases and disorders, organ transplantation rejection, graft-versus-host tissue diseases, and T-cell based lymphoma and leukemia.

### CROSS REFERENCE TO RELATED APPLICATIONS

This application claims benefit under 35 U.S.C.§119(e) of the U.S. Provisional application No. 60/927,107 filed May 1, 2007, the contents of which are incorporated herein by reference in its entirety.

### GOVERNMENT SUPPORT

This invention was made with Government support under grant No. AI054451 awarded by the National Institutes of Health. The Government has certain rights in the invention.

### FIELD OF INVENTION

The invention relates to methods of T cell immune system modulation and the treatment of immune system related diseases and disorders. Embodiments includes immunotherapeutics for the treatment of autoimmune diseases and disorders, organ transplantation rejection, graft-versus-host tissue diseases, and T-cell based lymphoma and leukemia. More particularly, the invention relates to antibodies, bioengineered antibodies and recombinant proteins that bind to T-cells and prevent T cell activation.

### BACKGROUND OF INVENTION

An immune system is a collection of mechanisms within an organism that protects against infection by identifying and killing foreign pathogens. It detects pathogens ranging from viruses to parasitic worms and distinguishes them from the organism's normal cells and tissues. Special cells derived from pluripotential hematopoietic stem cells in the bone marrow, the B- and T-lymphocytes, co-ordinate to recognize and destroy foreign pathogens as well as provide memory for recognizing the foreign pathogen after the initiation exposure. This cell-based memory ensures that the immune system can mount an rapid and robust defense response when the same foreign pathogen is encountered again in the future.

However sometimes the immune system fails to function properly and that leads to the disorders of the immune system: immunodeficiency, autoimmunity, and hypersensitivity. For example, in insulin-dependent diabetes mellitus (IDDM), T cells initiate the destruction of the insulin-producing beta cells of the islets of Langerhans in the pancreas. The chief culprits are CD4+ T cells of the inflammation-producing Th1 subset. In multiple sclerosis (MS), T cells, again mostly Th1 cells—initiate an attack that destroys the myelin sheath of neurons. As the disease progresses, other cells such as macrophages as well as antibodies participate in causing the damage. T cells react to antigens (as yet unidentified) in the joints and release tumor necrosis factor-alpha (TNF-α) with resulting inflammation and damage to the joints in rheumatoid arthritis (RA).

Rheumatoid arthritis (RA) is a disorder of chronic and systemic inflammation with a prevalence estimated at 1% of general population in North America. The key feature of RA is symmetrical joint inflammation and destruction driven by hypertrophic synnovial tissue called pannus, which causes erosion in joint cartilage and juxta-articular bone. Despite being the focus of numerous studies in the past, the pathogenesis of human RA is still poorly understood. Most of the knowledge has been gained from studies of murine models of inflammatory arthritis. The commonly used models include active collagen-induced arthritis (Courtenay, J. S., et. al., 1980, Nature 283:666), K/B×N mice (Kouskoff, V., et. al., 1996, Cell 87:811), and passive antibody-induced arthritis (Korganow, A. S., et. al., 1999, Immunity 10:451; Terato, K., et. al., 1992, J Immunol 148:2103). Thus far, published data have suggested that the development of inflammatory arthritis requires concerted actions of both adaptive and innate immune systems (Firestein, G. S. 2003, Nature 423:356). One key action of the adaptive immune system in the pathogenesis of inflammatory arthritis is the activation of CD4+ T (Th) cells. However, the arthritogenic antigen(s) that activates Th cells has yet to be identified. An arthritogenic antigen is any agent that stimulate the immune system to attack the joints, thereby cause inflammation and arthritis. Recent animal data actually indicate that even a ubiquitous auto-antigen (glucose-6-phosphate isomerase, GPI) can lead to joint inflammation (Matsumoto, I., et. al., 1999, Science 286:1732). It remains controversial whether GPI is indeed an arthritogenic antigen in humans (Matsumoto, I., et. al., 2003, Arthritis Rheum 48:944; Schaller, M., et. al., 2001, Nat Immunol 2:746.). Despite the limited progress in the search of arthritogenic antigens, it is believe that Th cells play a crucial role in the pathogenesis of inflammatory arthritis. Th cells are required for the development of joint inflammation in collagen-induced arthritis and K/B×N mice (Kouskoff, V., et. al., 1996, Cell 87:811). In addition, a spontaneous mutation in ZAP-70 was recently shown to alter thymic selection of Th cells and result in Th cell-mediated joint inflammation in mice (Sakaguchi, N., et. al., 2003, Nature 426:454; Hirota, K., et. al., 2007, J Exp Med 204:41). Human RA is strongly associated with a few HLA class II genes of the DRB1*0401 and *0404 types, leading to the hypothesis of shared epitope (Firestein, G. S., supra).

Once activated, Th cells provide help to B cells to mount humoral responses. Recent studies have suggested that B cells, by producing pathogenic antibodies, also contribute significantly to the development of arthritis in some animal models of arthritis. For example, when a T cell receptor (TCR) transgene recognizing bovine pancreas ribonuclease in the context of I-A^{k} was introduced into NOD mice, these mice (called K/B×N mice) spontaneously developed florid joint inflammation (Kouskoff, V., et. al., supra). The development of arthritis in these mice was dependent on Th and B cells, which aberrantly recognize GPI as antigen in the K/B×N mice (Korganow, A. S., et. al., supra, Matsumoto, I., et. al., supra). Surprisingly, transfer of serum obtained from K/B×N mice rapidly and reproducibly induced arthritis in recipient mice (Korganow, A. S., et. al., supra). Similarly, administration of monoclonal antibody against collagen can also induce arthritis in mice (Terato, K., et. al., supra). B cells probably also play a critical role in the propagation of joint inflammation in human RA. Depleting B cells with CD20 antibody (rituximab) has been shown to be effective even for RA patients who are refractory to anti-TNF treatment or methotrexate (Cohen, S. B., et. al., 2006, Arthritis Rheum 54:2793; Emery, P., et. al., 2006, Arthritis Rheum 54:1390).

The activation and recruitment of innate cells, including macrophages, mast cells, and neutrophils, to synovial tissue contributes to the inflammatory phase of arthritis. (Tanaka, D., et. al., 2006, Immunology 119:195; Wipke, B. T., and P. M. Allen, 2001, J Immunol 167:1601; Lee, D. M., et. al., 2002, Science 297:1689; Solomon, S., et. al., 2005, Eur J Immunol 35:3064). How innate immune cells are activated and recruited is still poorly understood. Data gathered from antibody transfer models of arthritis indicate that the complement system, activated by aggregated pathogenic antibodies, plays a key role in the activation and recruitment of innate immune cells (Ji, H., et. al., 2002, Immunity 16:157; Banda, N. K., et. al., 2006, J Immunol 177:1904). But the activation of the innate immune system can also take place without the participation of adaptive immunity. Activated innate immune cells as well as synoviocytes then secreted a large amount of inflammatory cytokines, such as TNF-α, IL-1, or IL-6, leading to the formation of pannus and erosion of cartilage and bone.

Although there are a number of treatments for these autoimmune disorders, the majority are geared toward inhibiting the anti-inflammatory responses that has been elicited by the activated T cells and these treatment have variable efficacy and side effects. Therefore alternative treatments are needed.

### SUMMARY OF THE INVENTION

The present invention is based on the discovery that antibodies made against the extracellular portion of the transmembrane protein itm2a expressed on CD4+ T cells can be used to ligate itm2a together. The ligation of cell surface itm2a prevents the activation of the T cell when the T cells are stimulated with anti-CD3 antibodies. Moreover, ligation of itm2a with anti-itm2a antibodies can also prevent the re-activation of previously stimulated, non naïve CD4+ T cells.

The present invention provides for an isolated antibody which specifically binds to the extracellular portion of the transmembrane protein itm2a (amino acids 87-263) or an amino acid fragment thereof, wherein the amino acid fragment is at least 8, 12, 16, 20 amino acids in length. The isolated antibody inhibits the activation of naïve CD4+ T lymphocytes and also inhibits the re-activation of previously challenged CD4+ T lymphocytes. The isolated antibody is a polyclonal, monoclonal, humanized, and chimeric antibody that bind specifically to the extracellular portion of the transmembrane protein itm2a from mouse or human. The polyclonal antibody can be raised in rabbits, mouse, chicken, goat, and donkey.

Embodied in the invention is an antibody fragment comprising an antigen binding region of an isolated antibody which specifically binds to the extracellular portion of the transmembrane protein itm2a. The antibody can be a Fab, F(ab)'2, Fv fragment, domain antibody (DAb) or functional variants thereof with comparable or greater itm2a binding capability.

In one embodiment, a recombinant protein comprising the antigen binding region of the isolated monoclonal antibody that binds to the extracellular portion of the transmembrane protein itm2a is provided.

In one embodiment, the invention provides hybridoma cell lines comprising a nucleic acid molecule that encoding an antibody that binds to the extracellular portion of the transmembrane protein itm2a, and also provides an isolated antibody produced by the hybridoma cell line.

In one embodiment, a host cell comprising a nucleic acid molecule encoding an antibody is provided. In another embodiment, a nucleic acid molecule encoding an antibody or amino acid fragment thereof is provided.

In one embodiment, a method of inhibiting naïve CD4+ T cell activation comprising contacting T cell with an effective amount of isolated itm2a antibody is provided. In another embodiment, a method of inhibiting previously challenged CD4+ T cell re-activation comprising said T cell with an effective amount of isolated itm2a antibody is provided.

Embodiments of the invention further include a method of treating a mammal with an auto-immune-related disease or disorder, the method comprising administering to the mammal a therapeutically-effective amount of itm2a antibody; a method of inhibiting organ transplantation rejection in a mammal comprising administering to the mammal a therapeutically-effective amount of itm2a antibody; a method of inhibiting graft-versus-host disease in a mammal comprising administering to the mammal a therapeutically-effective amount of itm2a antibody; a method of treatment of T-cells base lymphomas and leukemia in a mammal comprising administering to the mammal a therapeutically-effective amount of itm2a antibody; and a composition comprising an itm2a antibody and a pharmaceutically acceptable vehicle.

In one embodiment, the auto-immune-related disease or disorder is selected from a group consisting of rheumatoid arthritis, multiple sclerosis (MS), systemic lupus erythematosus, Graves' disease (overactive thyroid), Hashimoto's thyroiditis (underactive thyroid), Type 1 diabetes mellitus, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

In one embodiment, the T-cell based lymphomas and leukemia are selected from a group consisting of cutaneous T-cell lymphoma (CTCL), adult T-cell leukemia, T-prolymphocytic leukaemia (TPPL), T cell large granular lymphocytic leukemia, extranodal T cell lymphoma, nasal type, enteropathy-type T cell lymphoma, hepatosplenic T cell lymphoma, primary cutaneous CD30-positive T cell lymphoproliferative disorders such as primary cutaneous anaplastic large cell lymphoma and lymphomatoid papulosis, angioimmunoblastic T cell lymphoma, peripheral T cell lymphoma, and anaplastic large cell lymphoma.

In one embodiment, the isolated anti-itm2a antibody specifically binds to the extracellular portion of the transmembrane protein itm2a (amino acids 87-263) or an amino acid fragment thereof.

In one embodiment, the method of treating a mammal with an auto-immune-related disease or disorder further comprise administering an autoimmune disease and disorder therapy such as azathioprine, infliximab, omalizumab, daclizumab, adalimumab, eculizumab, efalizumab, natalizumab, and omalizumab.

In one embodiment, the method of inhibiting organ transplantation rejection and the method of inhibiting graft-versus-host disease further comprise administering in combination with an immunosurppressant therapy used in transplantation, such as methotrexate, cyclosporin, daclizumab, basiliximab, azathioprine, muromonab-CD3, and mycophenolate.

In one embodiment, the method of treatment of T-cells base lymphomas and leukemia further comprise administering in combination with a cancer therapy used in treating T-cell based lymphoma and leukemia.

### BRIEF DESCRIPTION OF THE DRAWINGS

FIG. 1. A schematic diagram of the itm2a protein.

FIG. 2. Comparison of the amino acid sequences between murine (SEQ. ID. NO: 1) and human (SEQ. ID. NO: 2) itm2a. The non-conserved residues are marked with asterisks.

FIG. 3A. Western blot showing the detection of itm2a expression in 293 transfected cell with itm2a antibody.

FIG. 3B. FACS analysis of gated on GFP+293 cells transfected with a GFP-expressing vector alone (none) or in combination with a vector expressing murine (m) Itm2a, Itm2b, Itm2b, or human (h) Itm2a and labeled with itm2a antibody.

FIG. 4A. FACS analysis showing the expression profile of itm2a in freshly isolated CD4+ T (Th) cells stimulated in vitro with plate bound anti-CD3 (1 μg/ml), soluble anti-CD28 (1 μg/ml), and IL-2 (50 unit/ml).

FIG. 4B. Western blot analysis showing itm2a expression in whole cell extract of FIG. 4A cells. Hsp90 antibody (loading controls).

FIG. 4C. FACS analysis showing the expression profiles of itm2a in previously stimulated but currently resting Th cells, and in re-stimulated (restimulated) Th-cells. The numbers indicate the percentages of cells stained positive for itm2a.

FIG. 5A. Graph showing the uptake of ³H-thymidine (T), as a measure of cell activation and proliferation, by freshly isolated CD4+ T cells that were unstimulated or stimulated with anti-CD3/anti-CD28/IL-2 in the presence of plate bound itm2a antibody or control IgG at the indicated concentrations.

FIG. 5B. Graph showing the secretion of IFN-γ by unstimulated or stimulated Th cells in the presence or absence (control IgG) of itm2a antibody.

FIG. 6. The FSC/SSC plots showing percentage of live cells in samples of Th cells unstimulated or stimulated with anti-CD3/anti-CD28 in the presence or absence (control IgG) of itm2a antibody.

FIG. 7A. Graph showing the uptake of ³H-thymidine (T), as a measure of cell activation and proliferation, by isolated and previously stimulated CD4+ T cells that have been re-stimulated again with anti-CD3 in the presence or absence of itm2a antibody.

FIG. 7B. FACS analysis showing the expression of CD69 in previously stimulated CD4+ T cells that have been re-stimulated again with anti-CD3 in the presence or absence of itm2a antibody.

FIG. 7C. Graph showing the production of IFN-γ in previously stimulated CD4+ T cells that have been re-stimulated again with anti-CD3 in the presence or absence of itm2a antibody.

FIG. 8A. FACS analysis showing the activation of Th cells as measured by an increase in size, in previously stimulated but currently resting Th cells that have been re-stimulated again with PMA/iono in the presence or absence of itm2a antibody.

FIG. 8B. FACS analysis showing the expression of CD25 in previously stimulated but currently resting Th cells that have been re-stimulated again with PMA/iono in the presence or absence of itm2a antibody.

FIG. 8C. Graph showing the production of IL-2 in previously stimulated CD4+ T cells that have been re-stimulated again with PMA/iono in the presence or absence of itm2a antibody.

FIG. 9. The FSC/SSC plots of the anti-CD3 or PMA/iono (P/I) re-stimulated Th cells shown in FIGS. 7 and 8. The percentages of cells in the circled live cell gates are indicated.

### DETAILED DESCRIPTION OF THE INVENTION

Unless otherwise explained, all technical and scientific terms used herein have the same meaning as commonly understood by one of ordinary skill in the art to which this disclosure belongs. Definitions of common terms in immunology, immune system related diseases and disorders, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); The ELISA guidebook (Methods in molecular biology 149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol 57, Jennie P. Mather and David Barnes editors, Academic Press, 1st edition, 1998) which are all incorporated by reference herein in their entireties.

Methods for the production of antibodies are well known in one skilled in the art, examples of which can are disclosed in PCT publication WO 97/40072 or U.S. Application. No. 2002/0182702, which are herein incorporated by reference in their entirety. The processes of immunization to elicit antibody production in a mammal, the generation of hybridomas to produce monoclonal antibodies, and the purification of antibodies may be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entireties; Brown, “Clinical Use of Monoclonal Antibodies,” in BIOTECHNOLOGY AND PHARMACY, pp. 227-49, Pezzuto et al. (eds.) (Chapman & Hall 1993).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### DEFINITIONS

As used herein, the term “ligate” means to tie or bind together. Itm2a antibodies have two ligand itm2a binding site. A single itm2a antibody can bind two separate itm2a molecules, one itm2a on one CD4+ T (Th) cell and the second itm2a molecule from the same or a different Th cell. In doing so, a single anti-itm2a antibody can bind or ligate to two different surface itm2a molecules.

As used herein, the term “antibody” or “immunoglobulin” refers to immunoglobulin molecules and immunologically active portions of immunoglobulin molecules, i.e., molecules that contain an antigen binding site that immunospecifically binds an antigen. The terms also refers to antibodies comprised of two immunoglobulin heavy chains and two immunoglobulin light chains as well as a variety of forms besides antibodies; including, for example, Fv, Fab, and F(ab)'2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2nd ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference.).

The terms “antigen” is well understood in the art and refer to the portion of a macromolecule which is specifically recognized by a component of the immune system, e.g., an antibody or a T-cell antigen receptor. The term “antigen” includes any protein determinant capable of specific binding to an immunoglobulin. Antigenic determinants usually consist of chemically active surface groupings of molecules such as amino acids or sugar side chains and usually have specific three dimensional structural characteristics, as well as specific charge characteristics.

As used herein, the term “isolated” is meant to describe a compound of interest (e.g., a antibody) that is in an environment different from that in which the compound naturally occurs. “Isolated” is meant to include compounds that are within samples that are substantially enriched for the compound of interest and/or in which the compound of interest is partially or substantially purified.

As used herein, the term “substantially enriched” or “purified” when used in reference to a compound of interest refers to the compound being present in a sample in greater concentration than it is found in nature. That is, the term does not imply absolute purity. Nonetheless, a compound that is substantially enriched or purified in a sample is generally present, for example, as comprising at least 50% of the compound of interest. The sample can have anywhere from at least 50%, at least 60%, at least 70%, at least 80%, at least 90%, at least 99%, and all the percentages between 50% to 100% of the compound of interest.

As used herein, the term “epitope” refers to a surface portion of an antigen capable of eliciting an immune response and also capable of binding specifically with the antibody produced to counter that response.

As used herein, the term “anti-itm2a antibody” or “itm2a antibody” refers to an isolated antibody which specifically binds to the extracellular portion of the transmembrane protein itm2a (amino acids 87-263) or an amino acid fragment thereof, wherein the amino acid fragment is at least 8 amino acids in length. The amino acid fragment of the exterior itm2a can also be 9-20 amino acids in length. The itm2a protein can be a mouse itm2a protein or human itm2a protein. The anti-itm2a antibody can also be an antibody fragment comprising an antigen binding region which specifically binds to the extracellular portion of the transmembrane protein itm2a. The fragment can be, for example, a Fab, F(ab)'2 Fv fragment or other antigen binding fragment (domain) of an antibody, e.g. a DAb. The antibody can be polyclonal, monoclonal, humanized, or chimeric antibody. The anti-itm2a antibody can also be a recombinant protein comprising the antigen binding region of an isolated monoclonal antibody that specifically binds to the extracellular portion of the transmembrane protein itm2a.

As used herein, the term “humanized” immunoglobulin or “humanized” antibody refers to an immunoglobulin comprising a human framework, at least one complementarity determining regions (CDR) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. For example, a humanized immunoglobulin would not encompass a chimeric mouse variable region/human constant region antibody.

As used herein, the term “framework region” refers to those portions of antibody light and heavy chain variable regions that are relatively conserved (i.e., other than the CDRs) among different immunoglobulins in a single species, as defined by Kabat, et al., op. cit. As used herein, a “human framework region” is a framework region that is substantially identical (about 85% or more) to the framework region of a naturally occurring human antibody.

As used herein, the term “chimeric” antibody refers to an antibody whose heavy and light chains have been constructed, typically by genetic engineering, from immunoglobulin gene segments belonging to different species. For example, the variable (V) segments of the genes from a mouse monoclonal antibody can be joined to human constant (C) segments, such as gamma1 and/or gamma4. A typical therapeutic or diagnostic chimeric antibody is thus a hybrid protein comprising at least one V region (e.g., VH or VL) or the entire antigen-binding domain (i.e., VH and VL) from a mouse antibody and at least one C (effector) region (e.g., CH(CH1, CH2, CH3, or CH4) or CL or the entire C domain (i.e., CH and CL) from a human antibody, although other mammalian species can be used. In some embodiments, especially for use in the therapeutic methods of the im2a antibodies should contain no CH2 domain.

The term “functional variants” as used herein refers to the antibody or fragments thereof that have amino acids mutations in the protein. The mutations results in comparable or greater itm2a binding of the parent protein.

As used herein, the term “treat” or “treatment” refer to both therapeutic treatment and prophylactic or preventative measures, wherein the object is to prevent or slow down (lessen) an undesired physiological change or disorder, such as reducing and ameliorating the symptoms associated with the disease or disorder, and slowing the development or spread of cancer. The term also means blocking the cellular effects that are causing the symptoms of the diseased and disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. Any of these treatment types or types of patients can also be excluded. For example, in treating autoimmune disease such as rheumatoid arthritis, the pain, swelling, and stiffness are reduced, and the number of activated T-cells inflammatory cells at the joints found at the affected joints are reduced, and rate of joint deformation is reduced. In treating T-cell base lymphoma and leukemia, there is a reduction in the rate or even cessation of aberrant T-cells proliferation. In preventing and/or treating organ transplant rejection and graft-versus-host disease, T cell-mediated immune reaction of the donor organ is reduced or prevented, there is reduce tissue damage caused by T-cell mediated immune reaction, and the organ/tissue is functioning at a minimum of 80% efficiency.

As used herein, the term “inhibiting T cell activation or re-activation” refers to preventing T-cell proliferation that T-cell receptor (TCR) dependent stimulation and also when T-ell proliferation that is independent of TCR stimulation, for example, in leukemias. Inhibition T cell activation or re-activation also means preventing, stopping the production and/or secretion of pro-activation and pro-proliferation cytokines such as IFN-γ, IL-4, IL-13, and TGF-13, and transcription factors.

As used herein, the term “inhibiting graft-versus-host disease” refers to preventing the T-cell activation and the T-cell mediate immune response to the donor graft which can lead to the destruction and/scaring of donated tissue such that the donated tissue cannot function or function at low efficiency in the host, at less than 50% efficiency. Inhibiting also refers to reducing and ameliorating the symptoms associated with the disease such as swelling, inflammation, pain, and fever.

As used herein, the term “effective amount” or “therapeutically effective amount” refers to an amount that can inhibit and prevent T-cell activation or re-activation, and also inhibit and prevent and/or cure the disease or disorder. The therapeutically effective amount can also lessen or results in the disappearance of symptoms, or cause the disease to go into remission.

Embodiments of the present invention are based on the discovery that antibodies made against the extracellular portion of the transmembrane protein itm2a can be used to ligate itm2a expressed on the surface of naïve CD4+ T cells. An itm2a antibody comprising of two heavy immunoglobulin chains and two light immunoglobulin chains has two antigen-binding regions. Each antigen-binding on a single antibody can bind an itm2a protein. Therefore a single itm2a antibody can bind two separate itm2a on the cell surface. The ligation of itm2a prevents the activation of naïve T cells when the T cells are stimulated with anti-CD3 antibodies. Moreover, the antibodies can also prevent the re-activation of previously stimulated, non naïve CD4+ T cells.

Itm2a is a type II integral transmembrane protein which is a member of the newly defined BRIOCHOS protein family (Sanchez-Pulido, L., et. al., 2002, Trends Biochem Sci 27:329), which includes itm2a, itm2b, itm2c, chondromodulin, and surfactant protein. All members of the BRICHOS family share a BRICHOS domain, which contains approximately 100 amino acid residues including two highly conserved cysteines. The itm2a gene contains 6 exons and is located on X-chromosome in both human and mouse (Pittois, K., et. al., 1999, Mamm Genome 10:54) and is highly conserved among mammals. Itm2a of human, mouse, and rat all contain 263 amino acid residues and the homology between human and mouse itm2a is greater than 95%. Itm2a was originally cloned as a marker gene of chondro-osteogenic differentiation (Deleersnijder, W., et. al., 1996, J Biol Chem 271:19475). It is expressed in 1-day-old fetal mandibular condyle explant cultures, which contain cells of chondrogenic and osteogenic lineages. The transcripts of itm2a are also enriched in skeletal muscle and the thymus but most of the organs/tissues express a negligible level of itm2a (Kirchner, J., and M. J. Bevan, 1999, J Exp Med 190:217, 39). Overexpression of itm2a has been shown to enhance myogenic differentiation and delay the expression of collagen type X in mouse ATDC5 cells during chondrogenic differentiation (Van den Plas, D., and J. Merregaert, 2004, Cell Biol. Int. 28:199; Biol. Cell 96:463). However, the function of itm2a has remained largely unknown.

Itm2a is also know as the E25A and BRICD2A. The mouse itm2a sequence is found in Genebank Accession No. NM 008409 (SEQ. ID. No. 1) and human itm2a gene sequence is found in Genebank Accession No. NM 004867 (SEQ. ID. No. 2). Both itma2a from the human and mouse have more than 80% identical amino acids.

The cellular role of itm2a in T cells was studies in Bevan et al in J. Exp. Med. 1999, 290:217-228. In this study, itm2a was identified as a gene that was induced by major histocompatibility complex-mediated positive selection of CD4 CD8 double positive (DP) thymocytes. DP thymocytes expressed a low level of itm2a, which was markedly induced after positive selection.

The inventors showed in their experiments that Itm2a is expressed at a very low level, if any at all, in naïve CD4+ T (Th) cells and its expression is induced within 24 hours after stimulation with anti-CD3. When the previously activated T cells were examined one week later, the surface level of itm2a is nearly undetectable but a significant amount of intracellular Itm2a is still present inside the cells. Upon re-stimulation with anti-CD3, the amount of total Itm2a is greatly increased but only a modest faction was expressed on the cell surface.

Unless otherwise indicated, all technical and scientific terms used herein have the same meaning as they would to one skilled in the art of the present invention.

One embodiment of the invention is an isolated antibody which specifically binds to the extracellular portion of the transmembrane protein itm2a (amino acids 87-263) or to an amino acid fragment thereof, wherein the amino acid fragment is at least 8 amino acids in length. The antigen used to immunize of a rabbit host to elicit an immune response and the production antibodies is the extracellular 176 amino acids of the itm2a protein. Alternatively, the antigen is an amino acid fragment thereof the extracellular 176 amino acids of the itm2a protein. The amino acid fragment is at least 8, 12, 16, or 20 amino acids in length. The itm2a protein is selected from mouse (SEQ. ID. No. 1) (Genebank Accession No. NM 008409) or from human (SEQ. ID. No. 2) (Genebank Accession No. NM 004867) as their extracellular domains are 95% identical in amino acid sequences (FIG. 2).

Encompassed in the invention is a recombinant protein representing the extracellular 176 amino acids of the itm2a protein or the amino acid fragments thereof. The amino acid fragments can be 8, 12, 16, or 20 amino acids in length, and can be used to induce antibodies according to methods well known in the art including, but not limited to, in vivo immunization, in vitro immunization, and phage display methods. See, e.g. Wilson et al., Cell 37:767 (1984) and Bittle et al., J. Gen. Virol., 66:2347-2354 (1985). If in vivo immunization is used, animals can be immunized with free peptide; however, anti-peptide antibody titer can be boosted by coupling the peptide to a macromolecular carrier, such as keyhole limpet hemacyanin (KLH) or tetanus toxoid. For instance, peptides containing cysteine residues can be coupled to a carrier using a linker such as maleimidobenzoyl-N-hydroxysuccinimide ester (MBS), while other peptides can be coupled to carriers using a more general linking agent such as glutaraldehyde.

The antibody of the invention immunospecifically recognizes a epitope of amino acid sequence in the extracellular 176 amino acids of the itm2a protein or the amino acid fragments thereof that are at least 8, 12, 16, 20 amino acids in length.

In one embodiment, the antibody of the invention is an antibody fragment comprising an antigen binding region which specifically binds to the extracellular portion of the transmembrane protein itm2a or to an amino acid fragment thereof, wherein the amino acid fragment is at least 8, 12, 16, 20 amino acids in length. The antigen binding region of the antibody fragment can be the Fab, F(ab)'2 Fv fragment or DAb fragment.

The isolated antibody and antibody fragments that bind specifically to the extracellular portion of itm2a is herein referred to as itm2a antibody.

In one embodiment, the isolated itm2a antibody is a polyclonal antibody. In one embodiment, the itm2a antibody is a monoclonal antibody. In preferred embodiment, the itm2a antibody is a humanized antibody. In preferred another embodiment the itm2a antibody is a chimeric antibody. In yet another embodiment, the isolated itm2a antibodies include, but are not limited to multispecific, human, single chain antibodies, Fab fragments, F(ab)'2 fragments, DAb fragment, fragments produced by a Fab expression library, domain-deleted antibodies (including, e.g., CH2 domain-deleted antibodies), anti-idiotypic (anti-Id) antibodies (including, e.g., anti-Id antibodies to antibodies of the invention), and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Itm2a antibodies of the invention also include, but are not limited to, engineered forms of antibodies and antibody fragments such as diabodies, triabodies, tetrabodies, and higher multimers of scFvs, single-domain antibodies, as well as minibodies, such as two scFv fragments joined by two constant (C) domains. See, e.g., Hudson, P. J. and Couriau, C., Nature Med. 9: 129-134 (2003); U.S. Publication No. 20030148409; U.S. Pat. No. 5,837,242 and these are incorporated hereby reference in their entirety.

In one embodiment, the itm2a antibodies of the invention can be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al, and this is incorporated hereby reference in its entirety.

In a preferred embodiment for use in humans, the itm2a antibodies are human or humanized antigen-binding antibody fragments of the present invention and include, but are not limited to, Fab, Fab and F(ab)'2, Fd, single-chain Fvs (scFv), single-domain antibodies, triabodies, tetrabodies, minibodies, domain-deleted antibodies, single-chain antibodies, disulfide-linked Fvs (sdFv) and fragments comprising either a variable light chain (VL) or variable heavy chain VH region. Antigen-binding antibody fragments, including single-chain antibodies, can comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains.

Preferred antibodies in the therapeutic methods of the invention are those containing a deletion of the CH2 domain.

In one embodiment, a chimeric antibody can contain at least the itm2a antigen binding Fab or F(ab)'2 region while the humanized antibody can contain at least the itm2a antigen binding Fv region fused to a human Fc region.

In preferred embodiments, the itm2a antibodies of the present invention (including molecules comprising, or alternatively consisting of, antibody fragments or functional variants thereof), immunospecifically bind to the extracellular portion of itm2a and do not cross-react with any other antigens.

In a preferred embodiment, the itm2a antibodies of the invention (including molecules comprising, or alternatively consisting of, antibody fragments or functional variants thereof) preferentially bind the extracellular portion of itm2a, or fragments thereof relative to their ability to bind other antigens.

In one embodiment, a hybridoma cell line comprising a nucleic acid molecule encoding an itm2a antibody is provided. In another embodiment, the isolated itm2a antibody produced by the hybridomal cell line is provided. Naïve BALB/c mice are immunized with the recombinant protein representing the extracellular portion of itm2a or amino acid fragments thereof in complete Freund's adjuvant. Alternatively, a transgenic animal that has been genetically modified to produce human antibodies, such as XENOMOUSE™ and HuMab mouse, or a transchromosome (TC) mouse, can be immunized to generate human itm2a polyclonal antibodies. Hybridoma cell lines, specific for itm2a protein, were prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, hybridoma cell lines were generated using standard PEG fusion of the non-secreting myeloma cells (P3×63.Ag8) to splenocytes overexpressing itm2a antibodies at a ratio 1:3 and selected in Hat (hypoxanthin, aminopterin, and thymidine) media in 96 well plates. After two weeks, individual supernatants were tested for reactivity with anti-itm2a activity by ELISA, Western blot, and immunohistochemistry. Positive hybridomas colonies were subcloned and screened for reactivity twice to ensure clonality. Antibodies were isolated from hybridoma supernatants by protein G affinity purification using standard methods.

From these hybridoma cell lines producing itm2a specific antibodies, the polynucleotides encoding the VL and VH regions of these antibodies can be cloned into cloning vectors such as TOPO vectors (Invitrogen Inc.) and used for further molecular biology manipulations to generate other chimeric and humanized antibodies, variant forms of itm2a antibodies, and recombinant itm2a-binding proteins.

In one embodiment, the present invention also provides antibodies that comprise, or alternatively consist of, functional variants (including derivatives) of the antibody molecules (e.g., the VH regions and/or VL regions) described herein, which antibodies immunospecifically binds the extracellular portion of itm2a or to an amino acid fragment thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an itm2a extracellular portion 176 amino acid polypeptide).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immuno specifically binds the extracellular portion of itm2a or to an amino acid fragment thereof,) can be determined using techniques described herein or by routinely modifying techniques known in the art.

Humanized immunoglobulins and human antibody variants of the invention have variable framework regions from a human immunoglobulin (termed an acceptor immunoglobulin), and CDRs substantially from the mouse or human itm2a VH and VL regions encoded by the selected hybridoma clones (referred to as the donor immunoglobulin). The constant region(s), if present, are also from a human immunoglobulin. The humanized antibodies and human antibody variants exhibit a specific binding affinity for the extracellular portion of itm2a of at least 10⁽²⁾, 10⁽³⁾, 10⁽⁴⁾, 10⁽⁵⁾, 10⁽⁶⁾, 10⁽⁷⁾, 10⁽⁸⁾, 10⁽⁹⁾, or 10⁽¹⁰⁾ M⁽⁻¹⁾.

The heavy and light chain variable regions of possible human acceptor antibodies are described by Kabat, Sequences of Proteins of Immunological Interest (National Institutes of Health, Bethesda, Md., 1987 and 1991). The human acceptor antibody is chosen such that its variable regions exhibit a high degree of sequence identity with those of the mouse itm2a antibody. The heavy and light chain variable framework regions can be derived from the same or different human antibody sequences. The human antibody sequences can be the sequences of naturally occurring human antibodies or can be consensus sequences of several human antibodies.

The design of humanized immunoglobulins can be carried out as follows. When an amino acid falls under the following category, the framework amino acid of a human immunoglobulin to be used (acceptor immunoglobulin) is replaced by a framework amino acid from a CDR-providing non-human immunoglobulin (donor immunoglobulin): (a) the amino acid in the human framework region of the acceptor immunoglobulin is unusual for human immunoglobulins at that position, whereas the corresponding amino acid in the donor immunoglobulin is typical for human immunoglobulins in that position; (b) the position of the amino acid is immediately adjacent to one of the CDRs; or (c) the amino acid is capable of interacting with the CDRs (see, Queen et al. WO 92/11018., and Co et al., Proc. Natl. Acad. Sci. USA 88, 2869 (1991), respectively, both of which are incorporated herein by reference). For a detailed description of the production of humanized immunoglobulins see, Queen et al. and Co et al.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

Other than for the specific amino acid substitutions discussed above, the framework regions of humanized immunoglobulins and human antibody variants are usually substantially identical, and more usually, identical to the framework regions of the human antibodies from which they were derived (acceptor immunoglobulin). Of course, many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin or human antibody variants.

The variable segments of humanized antibodies or human antibody variants produced as described supra are typically linked to at least a portion of an immunoglobulin constant region (Fc), typically that of a human immunoglobulin. Human constant region DNA sequences can be isolated in accordance with well-known procedures from a variety of human cells, such as immortalized B-cells (see Kabat et al., supra, and WO 87/02671). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

The humanized antibody or human antibody variants include antibodies having all types of constant regions, including IgM, IgG, IgD, IgA and IgE, and any isotype, including IgG1, IgG2, IgG3 and IgG4. When it is desired that the humanized antibody or human antibody variants exhibit cytotoxic activity, the constant domain is usually a complement-fixing constant domain and the class is typically IgG1. When such cytotoxic activity is not desirable, the constant domain can be of the IgG2 class. The humanized antibody or human antibody variants can comprise sequences from more than one class or isotype.

Chimeric itm2a antibodies of the inventions can comprise the VH region and/or VL region encoded by the nucleic acids of mouse or human itm2a antibody from the selected hybridoma cell line, and fused to the CH region and/or CL region of a another species, such as human or mouse or horse, etc. In preferred embodiments, a chimeric itm2a antibody comprises the VH and/or VL region fused to human C regions. The human CH2 domain can be deleted when antibodies are used in therapeutic purposes. Chimeric antibodies encompass antibody fragments, as described above.

The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (see Kabat et al., supra, and WO 87/02671). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Chimeric antibodies include antibodies having all types of constant regions, including IgM, IgG, IgD, IgA and IgE, and any isotype, including IgG1, IgG2, IgG3 and IgG4. When it is desired that the chimeric antibody exhibit cytotoxic activity, the constant domain is usually a complement-fixing constant domain and the class is typically IgG1. When such cytotoxic activity is not desirable, the constant domain can be of the IgG2 class. The chimeric antibody can comprise sequences from more than one class or isotype.

A variety of methods are available for producing such immunoglobulins. Because of the degeneracy of the genetic code, a variety of nucleic acid sequences encode each immunoglobulin amino acid sequence. The desired nucleic acid sequences can be produced by de novo solid-phase DNA synthesis or by PCR mutagenesis of an earlier prepared variant of the desired polynucleotide. All nucleic acids encoding the antibodies described in this application are expressly included in the invention.

Once expressed, the whole antibodies, their dimers, individual light and heavy chains, or other immunoglobulin forms of the present invention can be purified according to standard procedures in the art, including ammonium sulfate precipitation, affinity columns, column chromatography, gel electrophoresis and the like (see, generally, Scopes, R., Protein Purification, Springer-Verlag, N.Y. (1982), which is incorporated herein by reference). Substantially pure immunoglobulins of at least about 90 to 95% homogeneity are preferred, and 98 to 99% or more homogeneity most preferred, for pharmaceutical uses.

In one embodiment, the invention provides a recombinant protein comprising the antigen binding region of an isolated antibody that immunospecifically binds the extracellular portion of itm2a or to an amino acid fragment thereof, wherein the amino acid fragment is at least 8, 12, 16, 20 amino acids in length. This recombinant protein is hereby referred to as recombinant itm2a-binding protein. In one embodiment, the antigen binding region can include the Fab, F(ab)'2, Dab or Fv fragment of any itm2a antibody. The non-antigen binding region of the protein can contain cysteine residues for the dimerization of the recombinant itm2a-binding protein. In another embodiment, the recombinant protein can be multivalent, having several antigen-binding region in tandem in the polypeptide chain. The itm2a-binding recombinant protein can be conjugated to a detectable label, such as an enzyme, a fluorescent label, a luminescent label, or a bioluminescent label. The itm2a-binding recombinant protein can also be conjugated to a therapeutic or a liposome encapsulated with therapeutic agents, or a toxin e.g., a radioactive material.

In one embodiment, the itm2a antibody or fragments or functional variants thereof are coupled to a detectable label, such as an enzyme, a fluorescent label, a luminescent label, or a bioluminescent label. In another embodiment, the itm2a antibodies or fragments or functional variants thereof or the recombinant itm2a-binding protein that specifically bind the extracellular portion of itm2a are coupled to a therapeutic or a toxin, e.g., a radioactive material. In one embodiment, the antibodies and recombinant itm2a are coupled to a radioisotope.

In one embodiment, nucleic acid molecules encoding itm2a antibodies, antibody fragments or variants thereof, and recombinant itm2a-binding proteins are included. In another embodiment, host cells comprising the nucleic acid molecules encoding the itm2a antibodies, antibody fragments or variants thereof, and recombinant itm2a-binding proteins are also included.

In one embodiment, a nucleic acid molecule encodes an itm2a antibody (including molecules comprising antibody fragments or functional variants thereof) comprise a VH region having an amino acid sequence of any one of the VH regions encoded by a nucleic acid of the invention and a VL region having an amino acid sequence of any one of the VL regions encoded by a nucleic acid of the invention.

After selecting a hybridoma cell line that produces antibodies immunospecifically against the extracellular portion of itm2a, the nucleic acids of these hybridoma cells are harvested, the heavy chain and light chain mRNA are isolated, and the corresponding VH and VL sequence of the nucleic acid are amplified and determined, by any method known in the art.

The nucleic acids corresponding to the VH and VL regions of the selected itm2a antibody are cloned by PCR cloning into one or more suitable expression vectors. By way of non-limiting example, PCR primers including VH or VL nucleotide sequences, a restriction site, and a flanking sequence to protect the restriction site can be used to amplify the VH or VL sequences. Utilizing cloning techniques known to those of skill in the art, the PCR amplified VH regions can be cloned into vectors expressing the appropriate immunoglobulin constant region, e.g., the human IgG1 or IgG4 constant region for VH regions, and the human kappa or lambda constant regions for kappa and lambda VL regions, respectively. Preferably, the vectors for expressing the VH or VL regions comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL regions can also be cloned into a single vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art (See, for example, Guo et al., J. Clin. Endocrinol. Metab. 82:925-31 (1997), and Ames et al., J. Immunol. Methods 184:177-86 (1995) which are herein incorporated in their entireties by reference).

The nucleic acid sequences encoding the various itm2a antibodies can be used for transformation of a suitable mammalian or non mammalian host cells or to generate phage display libraries, for example. Additionally, polypeptide antibodies of the invention can be chemically synthesized or produced through the use of recombinant expression systems that are known in the art.

The process of cloning the cDNA sequence that encodes the extracellular region of itm2a (amino acid 87-263) or amino acid fragments thereof, the construction of the various itm2a expression vectors, the constructions of the chimeric antibody and humanized antibody expressing vectors, and the protein expression and purification of itm2a antibodies, recombinant itm2a-binding proteins, and the generation of antibodies having a different amino acid sequence, for example to create amino acid substitutions, deletions, and/or insertions, can be performed by conventional recombinant molecular biology and protein biochemistry techniques such as described in Maniatis et. al. (Molecular Cloning—A Laboratory Manual; Cold Spring Harbor, 1982) and DNA Cloning Vols I, II, and III (D. Glover ed., IRL Press Ltd.), Sambrook et. al., (1989, Molecular Cloning, A Laboratory Manual; Cold Spring Harbor Laboratory Press, NY, USA), Current Protocols in Molecular Biology (CPMB) (Fred M. Ausubel, et al. ed., John Wiley and Sons, Inc.) and Current Protocols in Protein Science (CPPS) (John E. Coligan, et. al., ed., John Wiley and Sons, Inc.) which are all incorporated by reference herein in their entireties.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., Science 242:1038-1041 (1988)).

The processes of immunization to elicit antibody production in a mammal, the generation of hybridomas to produce monoclonal antibodies, and the purification of antibodies can be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entireties.

The step of immunizing an animal for eliciting antibodies can include injecting the antigen directly into the animal. The animal can be a non-human mammal such as goats, mouse, donkey, sheep, and rabbit. For example, the antigen can be injected into a mouse to elicit polyclonal antibodies, or monoclonal antibodies by using a hybridoma technology. The animal can be a natural animal, a transgenic animal that has been genetically modified to produce human antibodies, such as XENOMOUSE™ and HuMab Mouse, or a transchromosome (TC) mouse.

Optionally, the step of immunizing the animal can include transfecting the animal with an expression vector encoding the antigen. For example, DNA sequence encoding the antigen can be inserted into a mammalian expression vector or a viral vector (e.g., retroviral, adenoviral, and adeno-associated viral vectors) and the resulting expression vector can be injected into the animal where the expression of the antigen by the vector elicits immune responses to the antigen. Antibodies can then be isolated from the serum of the animal and used to target the membrane protein for therapeutic or diagnostic purposes.

Production of Itm2a Antibodies and Recombinant Itm2a-Binding Protein

The antibodies of the invention can be produced by any method known in the art for the synthesis of antibodies, in particular, by chemical synthesis or preferably, by recombinant expression techniques.

The recombinant proteins of the invention can be produced by any method known in the art for the expression and purification of recombinant proteins.

Recombinant expression of an antibody of the invention, or fragment, derivative or analog thereof, (e.g., a heavy or light chain of an antibody of the invention or a single chain antibody of the invention), including a recombinant protein derived from the antibody antigen-binding region, requires construction of an expression vector containing a polynucleotide that encodes the antibody. Once a polynucleotide encoding an antibody molecule or a heavy or light chain of an antibody or portion thereof (preferably containing the heavy or light chain variable domain) of the invention has been obtained, the vector for the production of the antibody molecule can be produced by recombinant DNA technology using techniques well known in the art. Thus, methods for preparing a protein by expressing a polynucleotide containing an antibody-encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors can include the nucleotide sequence encoding the constant region of the antibody molecule (see, e.g., PCT publication WO 86/05807; PCT publication WO 89/01036; and U.S. Pat. No. 5,122,464) and the variable domain of the antibody can be cloned into such a vector for expression of the entire heavy or light chain. Methods for generating multivalent and bispecific antibody fragments are described by Tomlinson I. and Holliger P. (2000) Methods Enzymol, 326, 461-479 and the engineering of antibody fragments and the rise of single-domain antibodies is described by Holliger P. (2005) Nat. Biotechnol. September; 23(9):1126-36, and are both hereby incorporated by reference.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains can be co-expressed in the host cell for expression of the entire immunoglobulin molecule, as detailed below.

A variety of host-expression vector systems can be utilized to express the antibody molecules of the invention. Such host-expression systems represent vehicles by which the coding sequences of interest can be produced and subsequently purified, but also represent cells which can, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); Cockett et al., BioTechnology, 8:2 (1990)).

In bacterial systems, a number of expression vectors can be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified can be desirable. Such vectors include, but are not limited, to the E. coli expression vector pUR278 (Ruther et al., EMBO J., 2:1791 (1983)), in which the antibody coding sequence can be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 24:5503-5509 (1989)); and the like pGEX vectors can also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety. Expression of antibody fragments in Pichia pastoris is described by Holliger, P. (2002) Meth. Mol. Biol., 178:349-57, and is hereby incorporated by reference.

In an insect system, Autographa californica nuclear polyhedrosis virus (AcNPV) is used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

Recent advances in the large scale expression of heterologous proteins in the algae Chlamydomonas reinhardtii are described by Griesbeck C. et. al. 2006 Mol. Biotechnol. 34:213-33; Manuell A L et. al. 2007 Plant Biotechnol J. Eprint; Franklin S E and Mayfield S P, 2005, Expert Opin Biol Ther. February; 5(2):225-35; Mayfield S P and Franklin S E, 2005 Vaccine Mar 7; 23(15):1828-32; and Fuhrmann M. 2004, Methods Mol. Med. 94:191-5. Foreign heterologous coding sequences are inserted into the genome of the nucleus, chloroplast and mitochodria by homologous recombination. The chloroplast expression vector p64 carrying the most versatile chloroplast selectable marker aminoglycoside adenyl transferase (aadA), which confers resistance to spectinomycin or streptomycin, can be used to express foreign protein in the chloroplast. Biolistic gene gun method is used to introduce the vector in the algae. Upon its entry into chloroplasts, the foreign DNA is released from the gene gun particles and integrates into the chloroplast genome through homologous recombination.

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. See, e.g., Logan & Shenk, Proc. Natl. Acad. Sci. USA, 81:355-359 (1984). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, Bittner et al., Methods in Enzymol., 153:51-544 (1987)).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERY, BHK, Hela, COS, MDCK, NSO, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compounds that interact directly or indirectly with the antibody molecule.

A number of selection systems can be used, including but not limited to the herpes simplex virus thymidine kinase (Wigler et al., Cell, 11:223 (1977)), hypoxanthine-guanine phosphoribosyltransferase (Szybalska & Szybalski, Proc. Natl. Acad. Sci. USA, 48:202 (1992)), and adenine phosphoribosyltransferase (Lowy et al., Cell, 22:817 (1980)) genes can be employed in tk-, hgprt- or aprt-cells, respectively. Also, anti-metabolite resistance can be used as the basis of selection for the following genes: dhfr, which confers resistance to methotrexate (Wigler et al., Proc. Natl. Acad. Sci. USA, 77:357 (1980); O′Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418; Wu and Wu, Biotherapy, 3:87-95 (1991); Tolstoshev, Ann. Rev. Pharmacol. Toxicol., 32:573-596 (1993); Mulligan, Science, 260:926-932 (1993); and Morgan and Anderson, Ann. Rev. Biochem., 62:191-217 (1993); Can, 1993, TIB TECH 11(5):155-215); and hygro, which confers resistance to hygromycin (Santerre et al., Gene, 30:147 (1984)). Methods commonly known in the art of recombinant DNA technology can be routinely applied to select the desired recombinant clone, and such methods are described, for example, in Current Protocols in Molecular Biology, Ausubel et al., eds. (John Wiley & Sons, NY 1993); Kriegler, Gene Transfer and Expression, A Laboratory Manual (Stockton Press, NY 1990); and Current Protocols in Human Genetics, Dracopoli et al., eds. (John Wiley & Sons, NY 1994), Chapters 12 and 13; Colberre-Garapin et al., J. Mol. Biol., 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)).

The host cell can be co-transfected with two expression vectors of the invention, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes, and is capable of expressing, both heavy and light chain polypeptides. In such situations, the light chain should be placed before the heavy chain to avoid an excess of toxic free heavy chain (Proudfoot, Nature, 322:52 (1986); Kohler, Proc. Natl. Acad. Sci. USA, 77:2197 (1980)). The coding sequences for the heavy and light chains can comprise cDNA or genomic DNA.

Once an antibody molecule of the invention has been produced by an animal or recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

### Therapeutic Applications

Embodied in the invention is an isolated itm2a antibody that binds cell surface itm2a which can inhibit the activation of naïve CD4+ T lymphocytes. Similarly, an itm2a-binding recombinant protein, comprising of an antigen binding region of an itm2a antibody, can bind T cell surface expressed itm2a and inhibits the activation of naïve CD4+ T cells. The itm2a-binding recombinant protein is multivalent and/or a dimmer. Naïve CD4+ T cells express a small amount of itm2a on the cell surface. Application of an itm2a antibody or an itm2a-binding recombinant protein to these naïve CD4+ T cells ligate cell surface itm2a and as a result prevents these CD4+ T cells from being stimulated by an antigen. Accordingly, embodied in the invention is a method of inhibiting naïve CD4+ T cell activation comprising contacting the T cell with an effective amount of an isolated itm2a antibody and/or a itm2a-binding recombinant protein. The method can include a combination of several different itm2a antibodies wherein each itm2a antibody binds to a unique epitope of the extracellular portion of itm2a protein. The method can include a combination of several different itm2a-binding recombinant protein wherein each itm2a binding protein binds to a unique epitope of the extracellular portion of itm2a protein. The method can also include a combination of isolated itm2a antibody and itm2a-binding recombinant protein, wherein the antibody and protein have different itm2a binding epitopes. In one embodiment, the method comprises an itm2a antibody and/or itm2a-binding recombinant protein in combination with other T-cell activation suppression molecules, eg. anti-IL-4, anti-IL6 and IL-10.

Activation of CD4+ T cells occurs through the engagement of both the T cell receptor (TCR) and CD28 on the T cell by antigens presented by the major histocompatibility complex and B7 family members on the antigen presenting cells (APC), respectively. Both are required for production of an effective immune response. The engagement of both the TCR and CD28 with MHC II complex and CD80 and CD86 proteins on the APC lead to several complex cascades of events in the T cells which include but are no limited to calcium influx, calcium release from the endoplasmic reticulum, protein complexes formation, protein phosphorylation, increase gene transcription and protein synthesis, protein secretion, genomic replication, and cell division. Proteins that are produced upon T cell activation include other cell surface receptors such as OX40 and ICOS, transcription factors such as NFAT and IL-2. NFAT is a transcription factor, which activates the transcription of a pleiotropic set of genes, most notable, IL-2, a cytokine that further enhances the proliferation of activated T cells. The activation of T cell also leads to the secretion of pro-activation and pro-proliferation cytokines and chemokines.

While not wishing to be bound by theory, the binding of an anti-itm2a antibody to the cell surface itm2a expressed on a T-cell can lead to physically blocking that T-cell from interacting with the antigen presenting MHC II complex and CD80 and CD86 proteins on the APC, and passively block the initiation of the cascade of events associated with T-cell activation upon TCR interaction with APC. Alternatively, the itm2a antibody can ligate several itm2a molecules on the surface of T cells. This aggregation of itm2a molecules by itm2a antibody can initiate a negative signal in T cells, thereby actively inhibiting the cascades of events associated with T-cell activation. It is envisioned that contacting naïve CD4+ T cell with anti-itm2a antibody can inhibit T-cell interaction with APC, and result in little, reduced or slower T-cell proliferation, protein synthesis, cytokine secretion, and cell receptor production.

Encompassed in the invention is an isolated itm2a antibody or an itm2a-binding recombinant protein comprising of an antigen binding region of an itm2a antibody can also bind and inhibit the re-activation of previously challenged CD4+ T-cells. Previously challenged but currently quiescent CD4+ T cells express and display a small amount of itm2a on the cell surface although research shows that there is a store of itm2a protein intracellularly. Application of an itm2a antibody or an itm2a-binding recombinant protein to these CD4+ T cells ligate cell surface itm2a and also prevents these CD4+ T cells from being stimulated by an antigen a second time. Accordingly, embodied in the invention is a method of inhibiting previously challenged CD4+ T cell re-activation comprising contacting T cell with an effective amount of isolated itm2a antibody and/or a itm2a-binding recombinant protein. The cascade of cellular events associated with the reactivation of a previously activated but quiescent T-cell is similar to that of a naïve T-cell, except the events can occur faster and the secretion of cytokines can occur faster because there is a supply of cytokine filled vesicles in the previously activated T-cell. The method can include a combination of several different itm2a antibodies wherein each itm2a antibody binds to a unique epitope of the extracellular portion of itm2a protein. The method can include a combination of several different recombinant itm2a-binding proteins wherein each itm2a binding protein binds to a unique epitope of the extracellular portion of itm2a protein. The method can also include a combination of isolated itm2a antibody and itm2a-binding recombinant protein, wherein the antibody and recombinant protein have different itm2a binding epitopes. In one embodiment, the method comprises an itm2a antibody and/or itm2a-binding recombinant protein in combination with other T-cell activation suppression molecules, e.g. anti-IL-4 anti-IL6 and IL-10 molecule.

The ligation of itm2a on T cells strongly suppressed proliferation, upregulation of CD69, and cytokine production of previously activated Th cells. This ability of inhibiting previously activated T cells represents a significant and clinically relevant avenue for the treatment and intervention of T cell-mediated diseases and disorders. T cell-mediated diseases and disorders can, but are not limited to, include organ-specific auto-immune diseases such as inflammatory arthritis, type I diabetes mellitus, multiple sclerosis, psoriasis, inflammatory bowel diseases, and vasculitis; allergic inflammation such as allergic asthma, atopic dermatitis, and contact hypersensitivity; organ transplantation rejection; graft-versus-host disease; and T cell lymphomas and leukemia.

In one embodiment, a method of treating a mammal with an auto-immune-related disease or disorder is provided. The method comprising administering to the mammal a therapeutically-effective amount of an isolated itm2a antibody and/or an recombinant itm2a-binding protein.

The itm2a antibody or recombinant itm2a-binding protein can be used as a potent immunosuppressant to target naïve as well as activated T cells and can be particularly beneficial in the following categories of clinical settings such as organ-specific autoimmune diseases, such as inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM)

Reagents targeting T cells have been developed and used to treat several autoimmune diseases (see review in Nature Immunology 2007, 8:25-30) For example, CTLA-41 g (Abatacept) inhibits the activation of T cells and has been approved for treating rheumatoid arthritis. Natalizumab interferes with the migration of T cells and is used for treating multiple sclerosis.

In another embodiment, a method disclosed herein comprise inhibiting organ transplantation rejection in a mammal comprising administering to the mammal a therapeutically-effective amount of itm2a antibody and/or an recombinant itm2a-binding protein. Transplant rejection occurs when the immune system of the recipient of a transplant attacks the transplanted donor organ or tissue such as the heart, lungs, pancreas, liver, and kidneys. This is because a normal healthy human immune system can distinguish foreign tissues and attempts to destroy them, just as it attempts to destroy infective organisms such as bacteria and viruses.

Acute organ rejection is generally mediated by T cell responses to proteins from the donor organ which differ from those found in the recipient. The development of T cell responses first occurs several days after a transplant if the patient is not taking immunosuppressant drugs. Acute organ rejection is caused by mismatched human leukocyte antigens (HLA) antigens that are present on all cells. HLA antigens are polymorphic therefore the chance of a perfect match is extremely rare. The reason that acute rejection occurs a week after transplantation is because the T-cells involved in rejection must be activated first by the foreign HLA, then differentiate and the antibodies in response to the allograft must be produced before rejection is initiated. These activated T-cells cause the graft cells to lyse or they produce cytokines that recruit other inflammatory cells, eventually causing necrosis of donor tissue. Endothelial cells in vascularized grafts such as kidneys are some of the earliest victims of acute rejection. Damage to the endothelial lining is an early predictor of irreversible acute graft failure. The new organ is then incapable of working at full efficiency, and symptoms of rejection become apparent to the transplant recipient. These symptoms of rejection are very similar to the symptoms of organ failure.

Physicians skilled in the art can recognize and diagnose transplantation rejection. A biopsy of the transplanted organ can confirm that it is being rejected. Some of the signs and symptoms of rejection for specific organs are as follow:

Kidney Rejection-Fever over 38° C. or 100.4° F., decreased urine output, weight gain over 2 pounds per day, increased blood pressure, and pain over kidney.

Liver Rejection-Fever over 38° C. or 100.4°, fatigue, jaundice (yellowing of skin or eyes), darkening of urine, clay-colored stools, and pain over liver.

Pancreas Rejection-Fever over 38° C. or 100.4° F., increased blood sugars and pain over pancreas.

The risk of acute rejection is highest in the first 3 months after transplantation. With the development of powerful immunosuppressive drugs such as cyclosporin, tacrolimus and rapamycin, the incidence of acute rejection has been greatly decreased, however, organ transplant recipients can develop acute rejection episodes months to years after transplantation. Acute rejection episodes can destroy the transplant if it is not recognized and treated appropriately. Episodes occur in around 60-75% of first kidney transplants, and 50 to 60% of liver transplants. Untreated acute rejection leads to scarring and damage of the donor organ, which then require the recipient to undergo a second or third organ transplantation, and often set the stage for chronic rejections of grafts.

Accordingly, administration of a therapeutically-effective amount of itm2a antibody shortly before organ or tissue transplantation, or immediately after transplantation can prevent organ/tissue transplantation rejection from developing. Itm2a antibody can also be administered at the initial diagnosis of such transplantation rejection to stop and/or prevent the rejection from progress further, or to slow the rejection progression to buy time while searching/waiting for another suitable organ to become available for transplantation. It is also envisioned that the therapeutically-effective amount of itm2a antibody can be administered in conjunction with powerful immunosuppressive drugs such as cyclosporin, tacrolimus and rapamycin to suppress organ or tissue transplantation rejection.

One embodiment of the invention is a method of inhibiting graft-versus-host disease in a mammal comprising administering to the mammal a therapeutically-effective amount of an isolated itm2a antibody and/or an recombinant itm2a-binding protein. A therapeutically-effective amount of an isolated itm2a antibody and/or an recombinant itm2a-binding protein can be used to prevent and treat early onset graft-versus-host disease (GVHD).

GVHD is a common complication of allogeneic bone marrow transplantation in which functional immune cells in the transplanted marrow recognize the recipient as “foreign” and mount an immunologic attack. After bone marrow transplantation, T cells present in the graft, neither as contaminants or intentionally introduced into the host, attack the tissues of the transplant recipient after perceiving host tissues as antigenically foreign. The T cells produce an excess of cytokines, including tumor necrosis factor (TNF) alpha and interleukin-1 (IL-1). A wide range of host antigens can initiate graft-versus-host-disease, among them the human leukocyte antigens (HLAs). However, graft-versus-host disease can occur even when HLA-identical siblings are the donors. HLA-identical siblings or HLA-identical unrelated donors often have genetically different proteins (called minor histocompatibility antigens) that can be presented by MHC molecules to the recipient's T-cells, which see these antigens as foreign and so mount an immune response.

While donor T-cells are undesirable as effector cells of graft-versus-host-disease, they are valuable for engraftment by preventing the recipient's residual immune system from rejecting the bone marrow graft (host-versus-graft). Additionally, as bone marrow transplantation is frequently used to cure cancer, mainly leukemias, donor T-cells have proven to have a valuable graft-versus-tumor effect.

Classically, acute graft-versus-host-disease is characterized by selective damage to the liver, skin and mucosa, and the gastrointestinal tract. Newer research indicates that other graft-versus-host-disease target organs include the immune system (the hematopoietic system—e.g. the bone marrow and the thymus) itself, and the lungs in the form of idiopathic pneumonitis. Chronic graft-versus-host-disease damages the above organs, but also causes changes to the connective tissue (e.g. of the skin and exocrine glands).

Transfusion-associated graft versus host disease (TA-GvHD) is a rare complication of blood transfusion, in which the donor T lymphocytes mount an immune response against the recipient's lymphoid tissue. Donor lymphocytes are usually identified as foreign and destroyed by the recipient's immune system. However, in situations where the recipient is immunocompromised (inborn immunodeficiency, acquired immunodeficiency, malignancy), or when the donor is homozygous and the recipient is heterozygous for an HLA haplotype (as can occur in directed donations from first-degree relatives), the recipient's immune system is not able to destroy the donor lymphocytes. This can result in graft versus host disease.

Graft-versus-host-disease can largely be avoided by performing a T-cell depleted bone marrow transplant. These types of transplants result in reduced target organ damage and generally less graft-versus-host-disease, but at a cost of diminished graft-versus-tumor effect, a greater risk of engraftment failure, and general immunodeficiency, resulting in a patient more susceptible to viral, bacterial, and fungal infection. Methotrexate and cyclosporin are common drugs used for GVHD prophylaxis.

An isolated itm2a antibody and/or itm2a-binding recombinant protein of the present invention can be administered simultaneously during the bone marrow transplant or blood transfusion, or administered shortly after thereafter as a prophylaxis in preventing GVHD and TA-GvHD respectively. In one embodiment, the itm2a antibody can be administered in conjunction with the common drugs used for GVHD prophylaxis, such as methotrexate and cyclosporin.

In one embodiment, the method described herein is a treatment of T cell based lymphomas and leukemia in a mammal comprising administering to the mammal a therapeutically-effective amount of an isolated itm2a antibody and/or itm2a-binding recombinant protein. T cell based cancers are cancers that have T cells as the primary malignant neoplastic cells. The cancer can be in the form of solid tumors (lymphomas) made of lymphatic and reticuloendothelial tissues or aberrant circulating and migrating T-cells in the form of leukemia. Examples of T cell based lymphomas and leukemia, but not limited to, include cutaneous T-cell lymphoma (CTCL), adult T-cell leukemia, T-prolymphocytic leukaemia (TPPL), T cell large granular lymphocytic leukemia, extranodal T cell lymphoma, nasal type, enteropathy-type T cell lymphoma, hepatosplenic T cell lymphoma, primary cutaneous CD30-positive T cell lymphoproliferative disorders such as primary cutaneous anaplastic large cell lymphoma and lymphomatoid papulosis, angioimmunoblastic T cell lymphoma, peripheral T cell lymphoma, and anaplastic large cell lymphoma.

In one embodiment, the itm2a antibody or recombinant itm2a-binding protein is radiolabelled or in complex with a radioisotope, toxin, prodrug or liposomes. The itm2a antibody-conjugate or recombinant itm2a-binding protein-conjugate targets the radioisotope, toxin, prodrug or liposomes encapsuling therapeutic prodrug, drug, or toxin to the aberrant T cell.

Conjugation of cytotoxic drugs to antibodies to achieve a targeted therapeutic result is well known in the art. For example, it is known that methotrexate (MTX) can be conjugated to antibodies and some selective cytotoxicity has been observed. The itm2a antibody can be conjugated with the fungal toxin maytansinoid (DM-1). It is desirable to enhance the cytotoxicity of such conjugates by increasing the loading of the cytotoxic drug. However, multiple conjugation of individual drug molecules to an antibody eventually reduces its immunoreactivity, the effect being observed when more than about ten drug molecules are loaded.

It has also been proposed that the drug be conjugated to a polymeric carrier, which in turn may be conjugated to an antibody. This has the advantage that larger numbers of drug molecules can be carried to the target site. Use of polylysine as a polymer carrier was reported by Ryser et al., Proc. Natl. Acad. Sci. USA, 75:3867-3870, 1978. These authors found that only about 13 MTX per carrier could be loaded and immunoreactivity was poor. In addition, the high amine content of the polymer, largely in the form of charged ammonium groups, caused the conjugate to stick to normal cells and vitiated the selectivity of the cytotoxic effect.

Rowland, U.S. Pat. No. 4,046,722, discloses an antibody conjugate wherein a plurality of molecules of a cytotoxic agent are covalently bound to a polymer carrier of molecular weight 5,000-500,000, and the loaded carrier is covalently bound to an antibody by random attachment to pendant amine or carboxyl groups. Ghose et al., J. Natl. Cancer Inst., 61:657-676, 1978, discloses other antibody-linked cytotoxic agents useful for cancer therapy. Shih et al., U.S. Pat. No. 4,699,784 discloses site specific attachment of a methotrexate-loaded aminodextran to an antibody.

Targeted neutron-activated radiotherapy is described, e.g., in Goldenberg et al., Proc. Natl. Acad. Sci. USA, 81:560 (1984); Hawthorne et al., J. Med. Chem., 15:449 (1972); and in Goldenberg, U.S. Pat. Nos. 4,332,647, 4,348,376, 4,361,544, 4,468,457, 4,444,744, 4,460,459, and 4,460,561, and in related pending applications U.S. Ser. Nos. 609,607 (filed May 14, 1984) and 633,999 (filed Jul. 24, 1984), the disclosures of all of which are incorporated herein in their entireties by reference.

The aforementioned references disclose, inter alia, methods of incorporating Boron-10-containing addends into antibody conjugates using, e.g., coupling of a carborane (e.g., linked to a phenyldiazonium ion) to an antibody are which suitable for incorporation of a relatively low number of Boron-10 atoms. Typically, between 10 and 120 B-10 atoms have been attached to IgG before the immunoreactivity and yield of recovered product become unacceptably low, using the carborane-phenyldiazonium conjugation procedure. It is desirable to be able to target a large number of B-10 atoms to a tumor site or cancerous cell for effective therapy. Itma2a antibodies radiolabeled with indium-111 and iodine-123 can deliver radiation with relative specificity to the aberrant T cells.

Additional methods of conjugating radioisotope, toxin, prodrug to itm2a antibody are fully described in U.S. Pat. No. 5,851,527 and is incorporated hereby reference in its entirety.

The methods of treating auto-immune disease and disorders, inhibiting organ transplantation rejection, inhibiting graft-versus-host disease, and treating T cell based lymphomas and leukemia can include a combination of several different itm2a antibodies wherein each itm2a antibody binds to a unique epitope of the extracellular portion of itm2a protein. The methods can include a combination of several different recombinant itm2a-binding protein wherein each itm2a-binding protein binds to a unique epitope of the extracellular portion of itm2a protein. The method can also include a combination of isolated itm2a antibody and itm2a-binding recombinant protein, wherein the antibody and recombinant protein have different itm2a binding epitope.

In addition, the itm2a antibodies and recombinant itm2a-binding proteins can be used as alternative or adjunct treatments, or used in combination with currently available treatment options for the specific auto-immune disease and disorders (azathioprine, infliximab, omalizumab, daclizumab, adalimumab, eculizumab, efalizumab, natalizumab, and omalizumab), organ transplantation rejection (daclizumab, anti-IL-2 antibody, azathioprine, mycophenolate), graft-versus-host disease (methotrexate and ciclosporin), and T cell based cancers (vincristine, doxorubicin, cyclophosphamide, etoposide, bleomycin, mitoxantrone and prednisolone). The itm2a antibodies and recombinant itm2a-binding proteins can be modified for clinical uses.

### Therapeutic/Prophylactic Compositions and Administration

In one embodiment, the invention provides a composition comprising an itm2a antibody or a recombinant itm2a-binding protein and a pharmaceutically acceptable vehicle. An itm2a antibody or a recombinant itm2a-binding protein should be substantially purified (i.e., substantially free from substances that limit its effect or produce undesired side-effects). The composition can be a combination of several different itm2a antibodies wherein each itm2a antibody binds to a unique epitope of the extracellular portion of itm2a protein and a pharmaceutically acceptable vehicle. The composition can include a combination of several different recombinant itm2a-binding proteins wherein each itm2a-binding protein binds to a unique epitope of the extracellular portion of itm2a protein and a pharmaceutically acceptable vehicle. The composition can also include a combination of isolated itm2a antibody and itm2a-binding recombinant protein, wherein the antibody and recombinant protein have different itm2a binding epitope and a pharmaceutically acceptable vehicle. In one embodiment, the composition comprise of an itm2a antibody or a recombinant itm2a-binding protein and another therapeutic agent along with a pharmaceutically acceptable vehicle. The therapeutic agent can be a therapeutic agent for treating of autoimmune diseases and disorders, organ transplantation rejection, graft-versus-host tissue diseases, and T-cell based lymphoma and leukemia.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18^{th} Ed., Gennaro, ed. (Mack Publishing Co., 1990). Such compositions will contain a therapeutically effective amount of the itm2a antibodies or recombinant itm2a-binding proteins, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylaminoethanol, histidine, procaine, to name a few.

Various delivery systems are known in the art and can be used to administer itm2a antibodies or recombinant itm2a-binding proteins of the invention, e.g., encapsulation in liposomes, microparticles, microcapsules, recombinant cells capable of expressing the itm2a antibodies or recombinant itm2a-binding proteins, receptor-mediated endocytosis (see, e.g., Wu and Wu, J. Biol. Chem., 262:4429-4432 (1987)). The composition can be delivered in a vesicle, in particular a liposome (see, Langer, Science, 249:1527-1533 (1990); Treat et al., in Liposomes in the Therapy of Infectious Disease and Cancer, Lopez-Berestein and Fidler, eds. (Liss, New York 1989), pp. 353-365; Lopez-Berestein, ibid., pp. 317-327; see, generally, ibid.). Methods of introduction include, but are not limited to, intradermal, intramuscular, intraperitoneal, intravenous, subcutaneous, intranasal, epidural, and oral routes. The compositions can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local. In addition, it can be desirable to introduce the pharmaceutical compositions of the invention into the central nervous system by any suitable route, including intraventricular and intrathecal injection; intraventricular injection can be facilitated by an intraventricular catheter, for example, attached to a reservoir, such as an Omcana reservoir. Pulmonary administration can also be employed, e.g., by use of an inhaler or nebulizer, and formulation with an aerosolizing agent.

In one embodiment, it can be desirable to administer the pharmaceutical compositions of the invention locally to the area in need of treatment, for example, for localized autoimmune diseases such as type 1 diabetes mellitus, Hashimoto's thyroidits, Graves' disease, celiac disease, multiple sclerolsis, Guillain-Barre syndrome, Addison's disease, and Raynaud's phenomenon. This can be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein of the invention, care must be taken to use materials to which the protein does not absorb.

In one embodiment, the composition can be delivered in a controlled release system.

In one embodiment, a pump can be used (see Langer, supra; Sefton, CRC Crit. Ref. Biomed. Eng., 14:201 (1987); Buchwald et al., Surgery, 88:507 (1980); Saudek et al., N. Engl. J. Med., 321:574 (1989)). In another embodiment, polymeric materials can be used (see, Medical Applications of Controlled Release, Langer and Wise, eds. (CRC Press, Boca Raton, Fla. 1974); Controlled Drug Bioavailability, Drug Product Design and Performance, Smolen and Ball, eds. (Wiley, New York 1984); Ranger and Peppas, Macromol. Sci. Rev. Macromol. Chem., 23:61 (1983); see also Levy et al., Science, 228:190 (1985); During et al., Ann. Neurol., 25:35 1 (1989); Howard et al., J. Neurosurg., 7 1:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science, 249:1527-1533 (1990)).

The precise dose to be employed in the formulation will also depend on the route of administration, and the seriousness of the disease or disorder, and should be decided according to the judgment of the practitioner and each patient's circumstances. Effective doses can be extrapolated from dose-response curves derived from in vitro or animal model test systems.

For itm2a antibodies or recombinant itm2a-binding proteins, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight.

In further embodiments of the invention, the itm2a antibody or recombinant itm2a-binding protein is radiolabelled or in complex with a radioisotope, toxin, prodrug or liposomes. Combinations can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The itm2a antibody or recombinant itm2a-binding protein compositions of the invention can be administered alone or in combination with other therapeutic agents, including but not limited to, chemotherapeutic agents, steroidal and non-steroidal anti-inflammatories, conventional immunotherapeutic agents and cytokines. Combinations can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In a preferred embodiment, the itm2a antibodies or recombinant itm2a-binding proteins compositions of the invention are administered in combination with autoimmune disease and disorder therapies including, but not limited to, azathioprine, infliximab, omalizumab, daclizumab, adalimumab, eculizumab, efalizumab, natalizumab, and omalizumab.

In a preferred embodiment, the itm2a antibodies or recombinant itm2a-binding proteins compositions of the invention are administered in combination with T-cell based lymphoma and leukemia therapy including, but not limited to, daclizumab, vincristine, doxorubicin, cyclophosphamide, etoposide, bleomycin, mitoxantrone and prednisolone.

In a preferred embodiment, the itm2a antibodies or recombinant itm2a-binding proteins compositions of the invention are administered in combination with other immunosurpressants including, but not limited to, methotrexate, ciclosporin, daclizumab, basiliximab, azathioprine, muromonab-CD3, and mycophenolate.

This invention is further illustrated by the following example which should not be construed as limiting. The contents of all references cited throughout this application, as well as the figures and table are incorporated herein by reference.

### EXAMPLES

### Example 1

### Cloning and Expression of the Extracellular Portion of Itm2a

The was cloned into plasmid pTRC His with an biotinylation tag (Avitag) using a three way ligation as follows. The pTRC His plasmid was double digested using BamHI and XhoI. The extracellular portion of Itm2a was excised from an expression vector from the NcoI site to the terminal XhoI site (part of the expression vector). A linker coding for the biotinylation tag was made by annealing two oligonucleotides (sense-5′-GA TCC GGT CTG AAC GAC ATC TTC GAG GCT CAG AAA ATC GAA TGG CAC GAA ATT TAC-3′ (SEQ. ID. No. 3), antisense 5′-C ATG GTA AAT TTC GTG CCA TTC GAT TTT CTG AGC CTC GAA GAT GTC GTT CAG ACC G-3′ (SEQ. ID. No. 4)) which have BamHI and NcoI overhangs. The three DNA fragments were ligated, transformed into bacteria. The sequence was confirmed by DNA sequencing.

The resultant bacterial expression plasmid was transformed into the AVB101 strain of E. coli (Avidity, Denver, Colo.). Transformed cells were induced with 0.6 mM isopropyl-β-D-thiogalactoside (IPTG) overnight. Cells were then lysed and the protein was purified using a nickel resin column. The eluted protein was dialyzed against phosphate buffered saline (PBS) and used to immunize rabbits (Proteintech Group Inc, Chicago, Ill.). Rabbits are immunized with ovalbumin/complete Freund's adjuvant (CFA) and recombinant itm2a protein. The immunized serum was then affinity purified as follows.

Biotinylated itm2a protein was run over streptavidin beads (Sigma, St Louis, Mo.) and washed with PBS. The serum was then run over the immobilized itm2a, washed, then eluted with low pH. The eluted antibody was then dialyzed against PBS and concentrated.

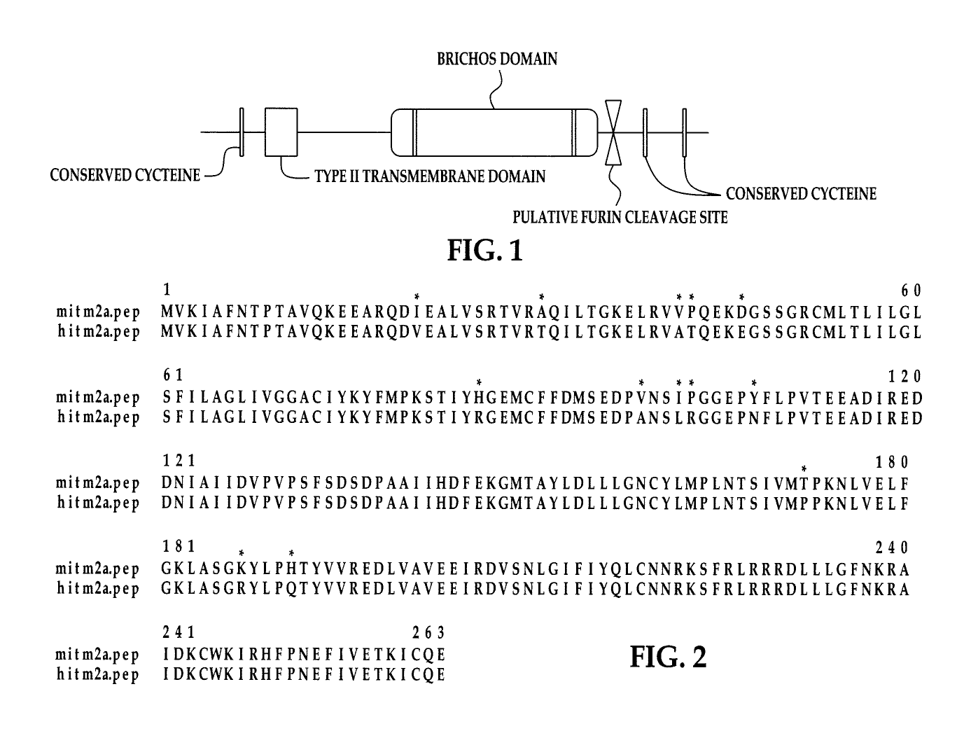

Itm2a is a type 2 integral transmembrane protein. It is a member of the newly defined BRICHOS protein family and closely related to Itm2b and Itm2c. The protein structure of Itm2a is shown in FIG. 1.

The Genbank ID for mouse itm2a is NM 008409 and for human itm2a is NM 004867.

The sequences of amino acid of Itm2a are highly conserved among mammals. Itm2a of human, rat, and mouse all contain 263 amino acid residues. The human and mouse itm2a are 95% homologous (FIG. 2).

The amino acid sequence of the extracellular portion of the murine itm2a that is used as the antigen to raised itm2a antibody is:

(SEQ. ID. No. 5)

GEMCFFDSEDPVNSIPGGEPYFLPVTEEADIREDDNIAIIDVPVPSFSDS
DPAAIIHDFEKGMTAYLDLLLGNCYLMPLNTSIVMTPKNLVELFGKLASG
KYLPHTYVVREDLVAVEEIRDVSNLGIFIYQLCNNRKSFRLRRRDLLLGF
NKRAIDKCWKIRHFPNEFIVETKICQE

### Example 2

### Generation of the Affinity-Purified Rabbit Polyclonal Itm2a Antibody

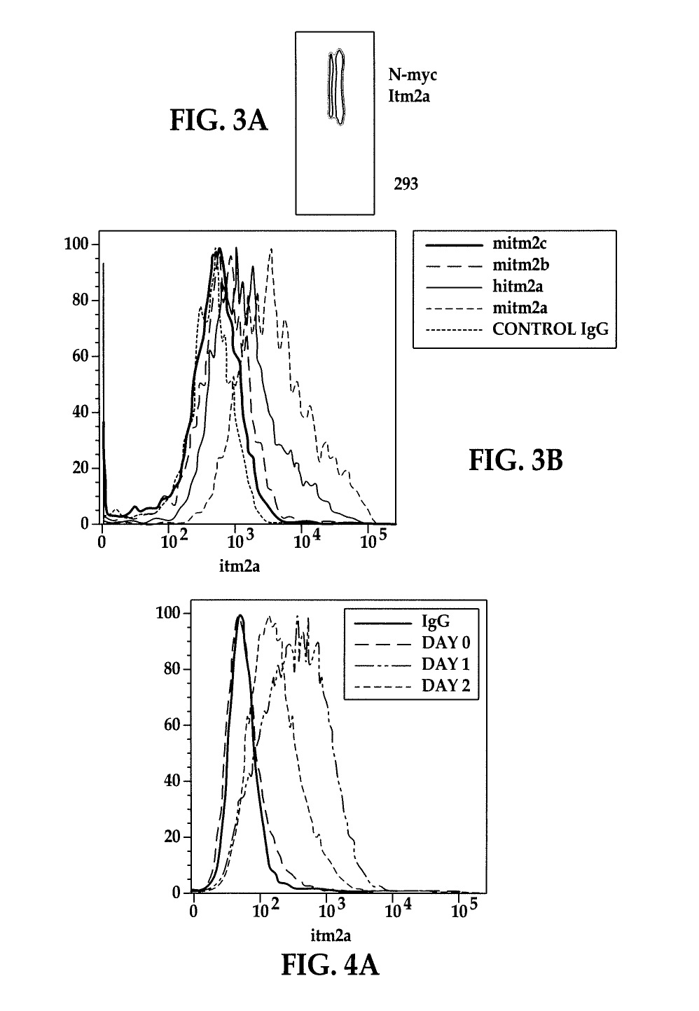

To facilitate the detection of itm2a, affinity-purified rabbit polyclonal antibody was raised against the extracellular portion of murine Itm2a, henceforth called itm2a antibody. 293 cells were transfected with an expression vector containing murine Itm2a or left untransfected. Whole cell lysate was harvested the next day, fractionated on a SDS-PAGE gel, transferred to a Hybond-N membrane, and probed with itm2a antibody. In a western blot, the itm2a antibody detected two protein bands in 293 cells transfected with an Itm2a-expressing vector but not in untransfected 293 cells (FIG. 3A). The Itm2a antibody, but not rabbit control IgG, also recognized cell surface Itm2a on 293 cells that were transfected with an itm2a-expressing vector but not a control plasmid (FIG. 3B).

The itm2a antibody is specific to Itm2a and cross-reacts to human Itm2a. 293 cells were transfected with a GFP-expressing vector along with a vector expressing murine (m) Itm2a, Itm2b, Itm2c, or human (h) Itm2a. On the next day, the 293 cells were stained with Itm2a antibody or rabbit control IgG followed by PE-conjugated goat anti-rabbit antibody. The stained cells were analyzed by FACS. The data shown are gated on GFP+ (transfected) cells. When the 293 cells were transfected with a plasmid vector expressing mouse itm2a, itm2b, or itm2c, only the itm2a-tranfected cells were recognized by the Itm2a antibody (FIG. 3B). In addition, the Itm2a antibody also recognized 293 cells transfected with a human itm2a-expressing vector (FIG. 3B), a result in agreement with the high degree of homology between human and mouse itm2a.

### Example 3

### Expression of Itm2a in Peripheral T Cells

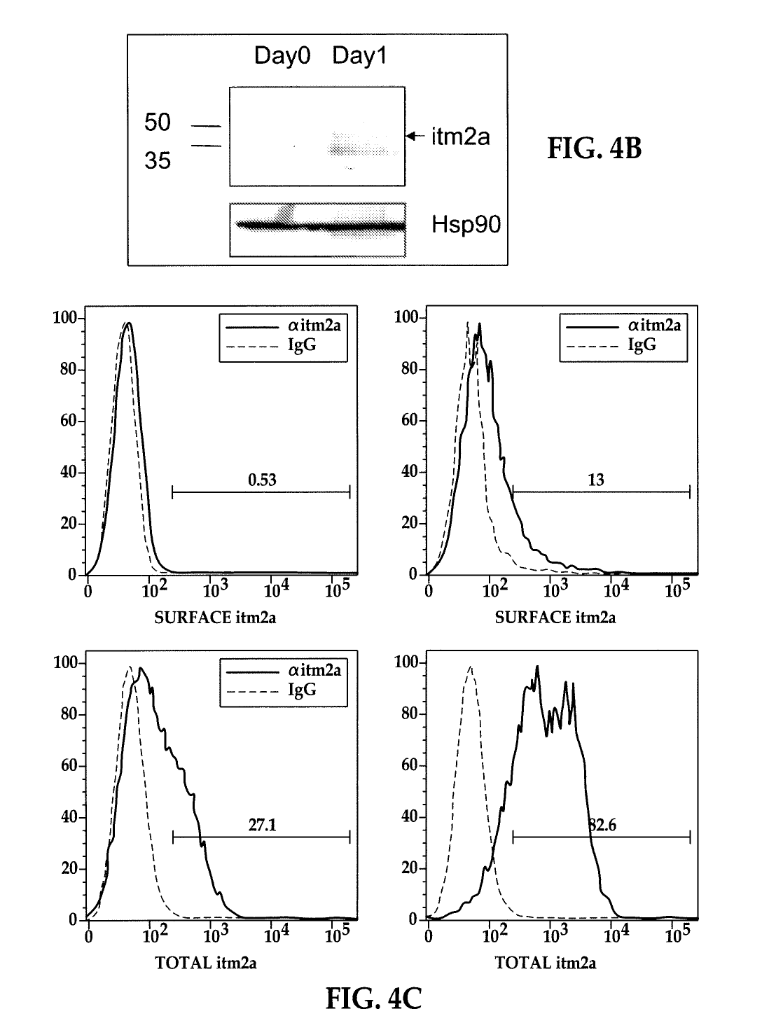

The itm2a antibody also recognized endogenous itm2a. Approximately 5-10% of thymocytes were stained positive for itm2a. The strongest staining (more than 40%) resided in the post-positive selection population (CD4+CD8+) (data not shown), a result consistent with the published data. Resting Th cells freshly isolated from mice expressed a low level of itm2a. Freshly isolated CD4+ T (Th) cells were stimulated in vitro with plate bound anti-CD3 (1 ug/ml), soluble anti-CD28 (1 ug/ml), and IL-2 (50 unit/ml). The Th cells were stained with itm2a antibody or control IgG (IgG) at the indicated time points according to the protocol described in FIG. 3B. Here the level of itm2a was rapidly induced when Th cells were activated by anti-CD3 (FIG. 4A). Similar to the result shown in FIG. 3A, two forms of endogenous itm2a were detected in activated Th cells by western blotting A (FIG. 4B), suggesting that itm2a can undergo either post-translational modifications or protease-mediated cleavages in Th cells. Whole cell extract was probed with itm2a antibody or Hsp90 antibody (loading controls) in a western blot. The positions of molecular weight markers of 35 and 50 kD are indicated. The expression of itm2a peaked within 24 hours after stimulation in most of the experiments but became nearly undetectable one week later.

The Th cells were rested for one week (resting) and a fraction of the Th cells were re-stimulated (restimulated) with plate bound anti-CD3 (1 ug/ml) for 24 hours. The cells were then analyzed for surface and total (after fixation and permeabilization) Itm2a expression. The numbers indicate the percentages of cells stained positive for itm2a.

When the Th cells were re-stimulated with anti-CD3, itm2a reappeared on the cell surface albeit at a relatively low level (FIG. 4C). Both CD4+ and CD8+ T cells were capable of expressing itm2a but the level of expression was slightly, but reproducibly higher in CD4+ T cells than that in CD8+ T cells.

It was reported that un-stimulated EL4 cells contained an easily detectable amount of intracellular itm2a which was translocated to the surface in response to stimulation with PMA/iono. In agreement with this observation, it was found that permeabilized Th cells always displayed brighter itm2a staining than unpermeabilized cells (FIG. 4C). For example, approximately 10% of freshly isolated Th cells were already positive for itm2a when the cells were stained after permeabilization (data not shown). At one week after stimulation, when there was very little itm2a on the surface of non-permeabilized cells, a substantial level (approximately 30% of the whole population) of itm2a was still detected in permeabilized cells. In addition, greater than 80% of re-stimulated Th cells contained itm2a when stained after permeabilization (FIG. 4C). As permeabilization allows itm2a antibody to detect both surface and intracellular itm2a, these results strongly indicate that peripheral Th cells contain both surface and intracellular itm2a and that the distribution of itm2a between the surface and intracellular compartments is highly dependent on the status and stage of activation. Taken together, the expression pattern of itm2a as determined with the itm2a antibody fully concurs with the published results, further confirming the fidelity of the itm2a antibody.

### Example 4

### Itm2a Antibody Inhibits the Activation of Freshly Isolated Naïve Th Cells

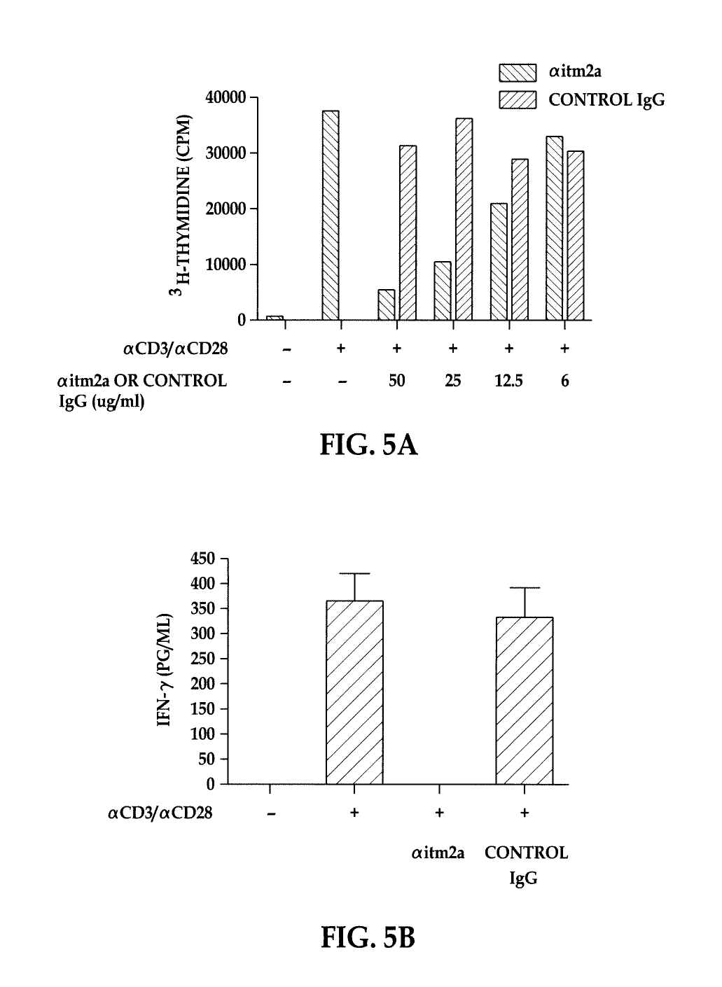

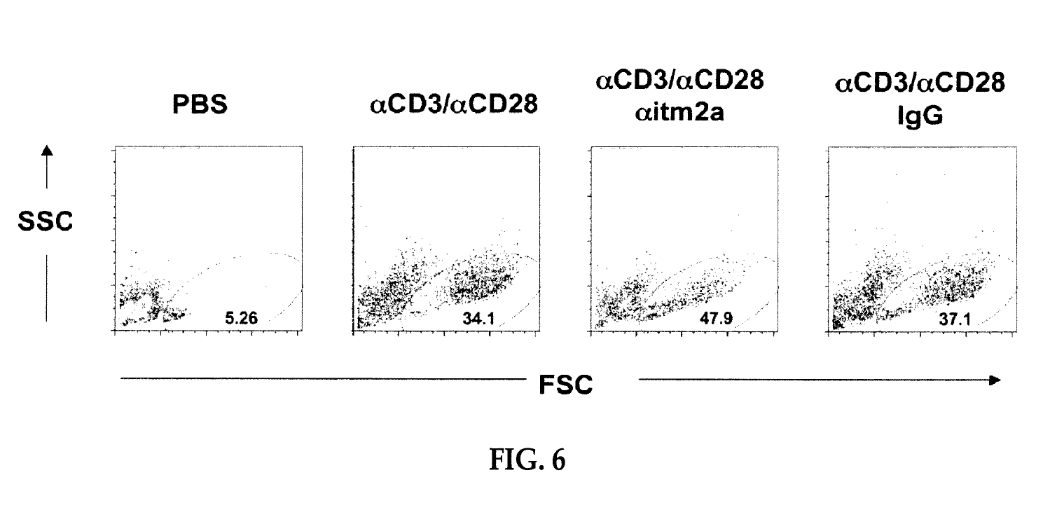

Itm2a is expressed on the surface of activated Th cells and is therefore subjected to interactions with other surface proteins expressed on neighboring cells. Such interactions can influence the activation of Th cells. It has been shown that ligation of transmembrane proteins by plate bound antibody is often sufficient to mimic or suppress the normal function of the transmembrane proteins. As the natural ligand of itm2a, if it does exist, is still unknown. The effects of ligation of itm2a with plate-bound itm2a antibody on the activation of Th cells were examined in this experiment. Freshly isolated CD4+ T cells were left unstimulated or stimulated in vitro in the presence of human IL-2 (50 units/ml) with plate bound anti-CD3 (1 ug/ml) and anti-CD28 (1 ug/ml) along with plate bound itm2a antibody or control IgG at the indicated concentrations (FIG. 5A) or at 50 ug/ml (FIG. 5B). The concentration of IFN-in supernatant was measured by ELISA 48 hours after stimulation (FIG. 5B). The cells were then pulsed with 3H-thymidine for 24 hours and the uptake of 3H-thymidine was determined (FIG. 5A). Two control antibodies were initially used (polyclonal rabbit IgG and affinity purified polyclonal rabbit anti-estrogen receptor) and yielded similar results. Subsequently the polyclonal rabbit IgG was used as controls in subsequent experiments. Both itm2a antibody and control IgG were dialyzed thoroughly against PBS prior to coating tissue culture plates (overnight at 4° C.). The antibody-coated plates were washed several times with PBS prior to use. Stimulated Th cells were analyzed 2-3 days later. As shown in FIG. 5A, itm2a antibody inhibited the uptake of ³H-thymidine by Th cells in a dose-dependent manner. A majority of the Th cells stimulated in the presence of itm2a antibody failed to undergo blastic change, upregulate CD69 and CD25, or produce cytokines, including IL-2, IL-4, and IFN-γ (FIG. 5B and data not shown). In contrast, plate bound control IgG had little effect on the activation of Th cells. One possible explanation for these observations is that itm2a antibody actively induces cell death, thereby preventing activation from happening. When Th cells were stimulated with anti-CD3/anti-CD28 (in the presence or absence of control IgG), approximately 35% of the cells fell into the live cell gate of FSC/SSC plot two days after stimulation, a time point when the stimulated cells were about to enter the active proliferation phase. Plate bound itm2a antibody had no negative effect on the percentage of live cells (47%) even though it strongly inhibited the activation of Th cells. In contrast, only 5% of the cells stayed alive after left un-stimulated for two days because most of the cell had undergone apoptosis-by-neglect. This observation suggests that itm2a antibody does not directly induce apoptosis. As shown in FIG. 6, the percentage of live cells remain approximately the same for Th cells were stimulated with anti-CD3/anti-CD28 (in the presence or absence of control IgG or itm2 antibody). Instead itm2a antibody probably attenuates activation signals to a level that is sufficient for escaping apoptosis-by-neglect but still inadequate for full-scale activation. As all features of activation are affected, itm2a antibody very likely acts on a proximal step of signaling cascade. Of note, itm2a antibody has a modest bioactivity and is suppressive only at a concentration greater than 12 ug/ml (FIG. 5A). Such modest bioactivity is not unexpected because the antibody is purified from rabbit polyclonal serum raised against denatured recombinant itm2a protein produced in bacteria. Only a fraction of the antibody is expected to bind to native itm2a and not every recognizable epitope of itm2a is functionally relevant.

### Example 5

### Itm2a antibody inhibits anti-CD3-mediated, but not PMA/iono-induced, re-activation of Th cells.

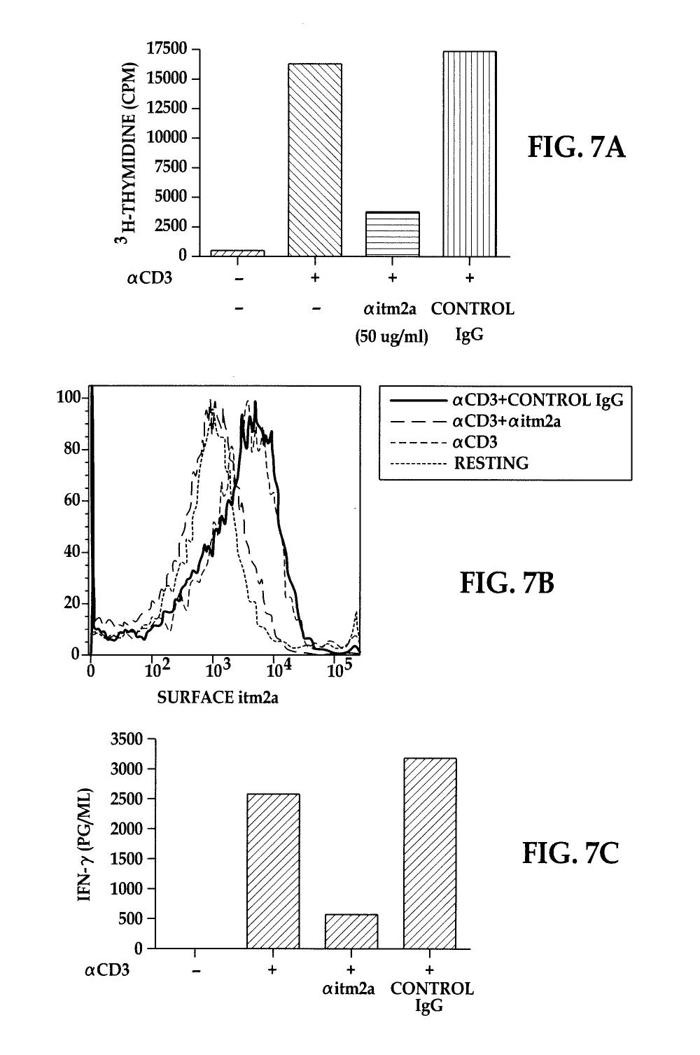

Itm2a is also expressed at a low level on the surface of previously activated Th cells and is further induced upon re-stimulation with anti-CD3 (FIG. 4C). Th cells were stimulated in vitro with anti-CD3/anti-CD28 in the presence of human IL-2 (50 units/ml). Seven days later, the stimulated Th cells were left unstimulated or restimulated with anti-CD3 (0.1 ug/ml) along with plate bound itm2a antibody (50 ug/ml) or control IgG. The expression of CD69 (FIG. 7B) and the production of IFN-(FIG. 7C) was examined 24 hours after re-stimulation. The cells were then pulsed with 3H-thymidine for 24 hours and the uptake of 3H-thymidine was measured (FIG. 7A).

To determine whether ligation of itm2a can also inhibit the re-activation of Th cells, freshly isolated Th cells was first stimulated with anti-CD3/anti-CD28 in the presence of IL-2. The Th cells were rested for 10-14 days and re-stimulated with plate bound anti-CD3 in the presence (or absence) of plate bound itm2a antibody or control antibody for 24 hours. It was found that itm2a antibody also markedly inhibited the re-activation of Th cells. There was a substantial reduction in proliferation, as measured by ³H-thymidine uptake (FIG. 7A), CFSE dilution assay (data not shown), the cell surface receptor CD69 expression (FIG. 7B) and the production of cytokine IL-2 (FIG. 7C). The blastic change, upregulation of CD25, and production of cytokines were nearly completely inhibited (FIGS. 7A-C and data not shown). In contrast, control antibody had no effect on anti-CD3-mediated re-activation of Th cells.

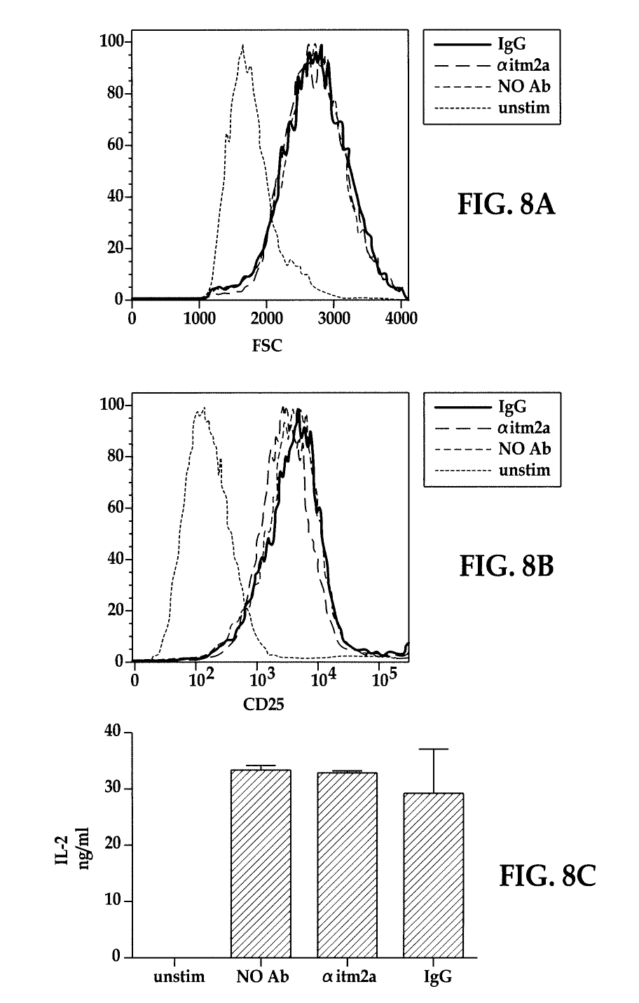

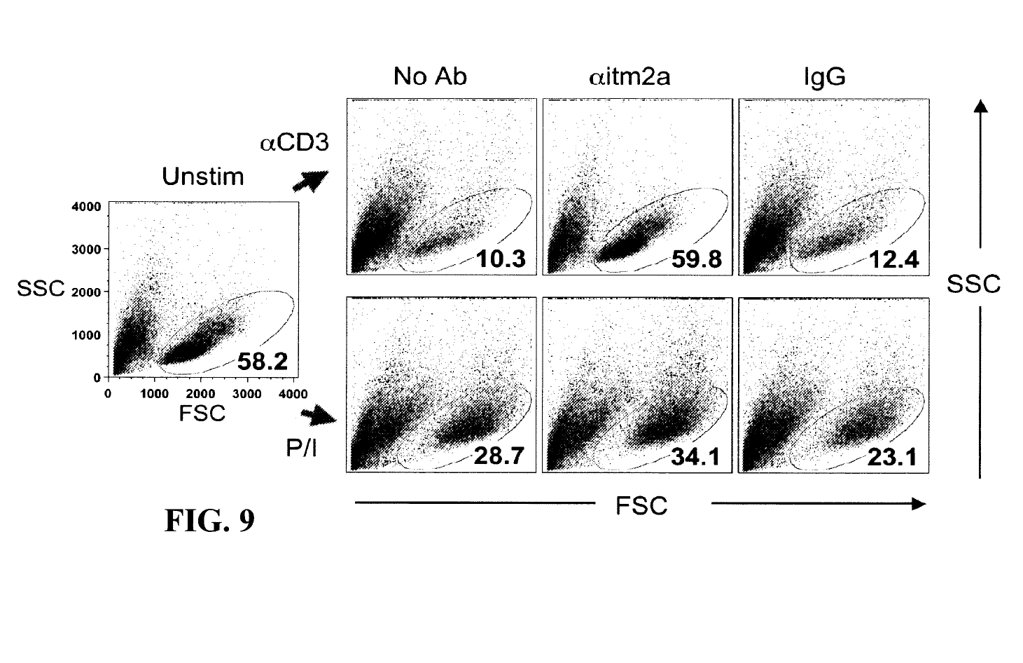

Surprisingly, plate bound itm2a antibody had no negative effect on the re-activation of Th cells induced by PMA/iono (FIGS. 8A-C). Th cells were stimulated in vitro with anti-CD3/anti-CD28 and human IL-2 (50 units/ml). 10-14 days later, the stimulated Th cells were left un-manipulated (unstim) or cultivated with plate-bound itm2a antibody (50 ug/ml), control IgG (IgG) or no antibody (no Ab) for one hour before the addition of PMA/iono. One day later, the cells were analyzed by FACS. The FSC and CD25 levels were shown in FIGS. 8A and B, respectively. The levels of IL-2 in supernatant were measured by ELISA and shown in FIG. 8C. The FSC/SSC plots of the anti-CD3 or PMA/iono (P/I) re-stimulated Th cells are shown FIG. 9. The FSC, CD25 and IL-2 levels were not affected by itm2a antibody when the Th cells were stimulated by PMA/iono.

As PMA/iono directly activates protein kinase C and calcium flux, this observation further supports the notion that itm2a antibody acts on an early signaling event that is bypassed by PMA/iono. In the absence of any stimulation greater than 50% of cells were in the live cell gate (FIG. 9). Re-stimulation with either anti-CD3 or PMA/iono caused activation-induced cell death (AICD) and resulted in fewer cells in the gate, an expected result of re-stimulating Th cells. The percentage of live cells however remained unchanged (59%) when the Th cells were stimulated with anti-CD3 in the presence of itm2a antibody. This result indicates that itm2a antibody inhibits the re-activation of Th cells, thereby preventing them from undergoing AICD, and that itm2a antibody does not actively induce cell death. Taken together these observations also argue strongly indicate the existence of non-specific toxic effect of plate bound itm2a antibody on Th cells.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method of inhibiting a naive CD4+ T cell activation comprising contacting a naïve CD4+ T cell with an effective amount of isolated anti-itm2a antibody.

19. A method of inhibiting a previously challenged CD4+ T cell re-activation comprising said T cell with an effective amount of isolated anti-itm2a antibody.

20. A method of treating a mammal with an auto-immune-related disease or disorder, said method comprising administering to said mammal a therapeutically-effective amount of isolated anti-itm2a antibody.

21. The method of claim 18, 19 or 20, wherein the isolated anti-itm2a antibody specifically binds to the extracellular portion of the transmembrane protein itm2a (amino acids 87-263) or an amino acid fragment thereof.

22. The method of claim 20, wherein the auto-immune-related disease or disorder is selected from a group consisting of rheumatoid arthritis, multiple sclerosis (MS), systemic lupus erythematosus, Graves' disease (overactive thyroid), Hashimoto's thyroiditis (underactive thyroid), Type 1 diabetes mellitus, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

23. The method of claim 20, further comprising administrating in combination with an autoimmune disease and disorder therapy.

24. The method of claim 23, wherein the autoimmune disease and disorder therapy is selected from azathioprine, infliximab, omalizumab, daclizumab, adalimumab, eculizumab, efalizumab, natalizumab, and omalizumab.

25. A method of inhibiting organ transplantation rejection in a mammal comprising administering to said mammal a therapeutically-effective amount of an isolated anti-itm2a antibody.

26. A method of inhibiting graft-versus-host disease in a mammal comprising administering to said mammal a therapeutically-effective amount of anti-itm2a antibody.

27. The method of claim 25 or 26, wherein the isolated anti-itm2a antibody specifically binds to the extracellular portion of the transmembrane protein itm2a (amino acids 87-263) or an amino acid fragment thereof.

28. The method of claim 25 or 26, further comprising administrating in combination with an immunosurppressant therapy used in transplantation.

29. The method of claim 28, wherein the immunosurppressant therapy is selected from methotrexate, cyclosporin, daclizumab, basiliximab, azathioprine, muromonab-CD3, and mycophenolate.

30. A method of treatment of T-cell based lymphomas and leukemia in a mammal comprising administering to said mammal a therapeutically-effective amount of anti-itm2a antibody.

31. The method of claims 30, wherein the isolated anti-itm2a antibody specifically binds to the extracellular portion of the transmembrane protein itm2a (amino acids 87-263) or an amino acid fragment thereof.

32. The method of claim 30, wherein the T-cell based lymphomas and leukemia are selected from a group consisting of cutaneous T-cell lymphoma (CTCL), adult T-cell leukemia, T-prolymphocytic leukaemia (TPPL), T cell large granular lymphocytic leukemia, extranodal T cell lymphoma, nasal type, enteropathy-type T cell lymphoma, hepatosplenic T cell lymphoma, primary cutaneous CD30-positive T cell lymphoproliferative disorders such as primary cutaneous anaplastic large cell lymphoma and lymphomatoid papulosis, angioimmunoblastic T cell lymphoma, peripheral T cell lymphoma, and anaplastic large cell lymphoma.

33. The method of claim 30, further comprising administrating in combination with a cancer therapy used in treating T-cell based lymphoma and leukemia.

34. The method of claim 33, wherein the cancer therapy is selected from daclizumab, vincristine, doxorubicin, cyclophosphamide, etoposide, bleomycin, mitoxantrone and prednisolone.

35. The method of claim 20, 25, 26, or 30, wherein the mammal is a human.


SEQUENCE LISTING (5 sequences)

SEQ ID NO: 1 - protein, 263 aa, Mus musculus
MVKIAFNTPTAVQKEEARQDIEALVSRTVRAQILTGKELRVVPQEKDGSSGRCMLTLLGLSFILAGLIVGGACIYKYFMPKSTIYHGEMCFFDSEDPVNSIPGGEPYFLPVTEEADIREDDNIAIIDVPVPSFSDSDPAAIIHDFEKGMTAYLDLLLGNCYLMPLNTSIVMTPKNLVELFGKLASGKYLPHTYVVREDLVAVEEIRDVSNLGIFIYQLCNNRKSFRLRRRDLLLGFNKRAIDKCWKIRHFPNEFIVETKICQE

SEQ ID NO: 2 - protein, 263 aa, Homo sapiens
MVKIAFNTPTAVQKEEARQDVEALLSRTVRTQILTGKELRVATQEKEGSSGRCMLTLLGLSFILAGLIVGGACIYKYFMPKSTIYRGEMCFFDSEDPANSLRGGEPNFLPVTEEADIREDDNIAIIDVPVPSFSDSDPAAIIHDFEKGMTAYLDLLLGNCYLMPLNTSIVMPPKNLVELFGKLASGRYLPQTYVVREDLVAVEEIRDVSNLGIFIYQLCNNRKSFRLRRRDLLLGFNKRAIDKCWKIRHFPNEFIVETKICQE

SEQ ID NO: 3 - DNA, 56 bp, Artificial Sequence
  Description of Artificial Sequence Synthetic oligonucleotide
gatccggtctgaacgacatcttcgaggctcagaaaatcgaatggcacgaaatttac

SEQ ID NO: 4 - DNA, 56 bp, Artificial Sequence
  Description of Artificial Sequence Synthetic oligonucleotide
catggtaaatttcgtgccattcgattttctgagcctcgaagatgtcgttcagaccg

SEQ ID NO: 5 - protein, 177 aa, Mus musculus
GEMCFFDSEDPVNSIPGGEPYFLPVTEEADIREDDNIAIIDVPVPSFSDSDPAAIIHDFEKGMTAYLDLLLGNCYLMPLNTSIVMTPKNLVELFGKLASGKYLPHTYVVREDLVAVEEIRDVSNLGIFIYQLCNNRKSFRLRRRDLLLGFNKRAIDKCWKIRHFPNEFIVETKICQE